(19) United States
(12) Patent Application Publication
(10) Pub. No.: US 2013/0202681 A1
(43) Pub. Date: Aug. 8, 2013

(54) LIPOSOMALLY ENCAPSULATED REDUCED GLUTATHIONE FOR MANAGEMENT OF CANCER AND DISRUPTION OF CANCER ENERGY CYCLES

(71) Applicant: Frederick Timothy Guilford, Palo Alto, CA (US)

(72) Inventor: Frederick Timothy Guilford, Palo Alto, CA (US)

(21) Appl. No.: 13/734,973

(22) Filed: Jan. 5, 2013

Related U.S. Application Data
(60) Provisional application No. 61/749,250, filed on Jan. 4, 2013.
(60) Provisional application No. 61/583,388, filed on Jan. 5, 2012.

(30) Foreign Application Priority Data
Jan. 5, 2013 (US) PCT/US2013/020422

(51) Int. Cl. A61K 9/127; A61K 31/198; A61K 38/06

(52) U.S. Cl. CPC A61K 9/127; A61K 38/063; A61K 31/198
USPC 424/450; 514/21.9; 514/4.8; 514/19.3

(57) ABSTRACT

A method of treatment of cancer with a formulation of liposomally encapsulated glutathione, that is preferably used orally, increases the level of glutathione in tissues in order to prevent and reverse the metabolic changes in cells that results in the formation of the metabolic “fuel supply” that supports cancer cells, and without which the cells can die out. The method prevents the oxidative stress that damages normal support cells such as stromal fibroblast cells. By blocking the “fuel supply,” the invention can protect, prevent and reverse these cells from the steps of autophagy and mitophagy, that results in the cells decreasing the normal production of ATP for energy and using aerobic glycolysis for energy production. The use of oral liposomally encapsulated glutathione will maintain the presence and normal function of caveolin in fibroblast and other cells, thus preventing their conversion to autophagic tumor stromal cells.

### FIELD OF INVENTION

This invention relates to treatment of cancer using reduced glutathione encapsulated in a liposome in a particular way, including in combination with other therapies and a related test for cancer biomarkers.

### SUMMARY OF INVENTION

This invention is intended to propose an agent to block the “fuel supply” that energizes cancer cell growth by protecting surrounding cells to the cancer, particularly stromal fibroblast cells. The invention disables the products of surrounding cells useable for energy conversion by the cancer cell thereby crippling the cell and disabling its growth process.

To state this in more detail, this application describes the use of a formulation of liposomally encapsulated glutathione that is preferably used orally to increase the level of glutathione in tissues in order to prevent and reverse the metabolic changes in cells that results in the formation of the metabolic fuel that supports cancer cells. Liposomally encapsulated glutathione prevents the oxidative stress that damages normal support cells such as fibroblasts and can prevent and reverse these cells from the steps of autophagy and mitophagy that results in the cells decreasing the normal mitochondrial production of ATP for energy and resorting to the use of aerobic glycolysis for energy production. The use of oral liposomally encapsulated glutathione will maintain the presence and normal function of caveolin in fibroblast and other cells, thus preventing their conversion to autophagic tumor stromal cells. By stopping the formation of autophagic cells, the production of the metabolic fuel needed by cancer cells is stopped, which results in the death of the cancer cells. The use of oral liposomally encapsulated glutathione provides a surprisingly high level of glutathione in tissues, which by decreasing the energy that the cancer cell has available to resist the cancer killing effects of chemotherapy agents, will result in enhanced susceptibility of cancer cells to chemotherapy agents. The lowered resistance of the autophagic cancer cells will allow the use of lower doses of chemotherapy agent. The increased demand for glucose in the autophagic stromal cells will allow the use of insulin potentiated therapies that selectively target cells with a high glucose requirement.

Targeting the increased use of glucose by the autophagic stromal cells surrounding cancer cells with agents that disrupt glycolysis such as dichloracetic acid (DCA) will also decrease the fuel sources for cancer cells. Macrophages undergoing oxidative stress lose the ability to form nitric oxide (NO) efficiently and instead increase the use of the arginase enzyme, a metabolism that goes on to form a type of macrophage that is supportive of cancer cell growth. Liposomally encapsulated glutathione will prevent the formation of macrophages which have become tumor supportive and will restore to normal the tumor-cidal activity of macrophages. It has also been observed that toxins from molds common to the environment are often associated with tumor tissue and may have compromised the physiology of macrophages in such a way that they have become reservoirs for the mold or fungal metabolism. In effect, the macrophage may become transformed to caveolin positive cancer cells. The physiology related to the formation of autophagic stromal cells can be monitored with a combination of biomarkers that monitor serum levels of caveolin-1, C-reactive protein and oxidized LDL cholesterol.

### BACKGROUND

In spite of advancement in cancer surveillance and therapy, cancer remains a leading cause of death. It is estimated that 1,529,560 men and women (789,620 men and 739,940 women) will be diagnosed with cancer and 569,490 men and women will die of cancer (including all of the sites cataloged in the body for cancer) in 2010, according to the National Cancer Institute (1). It is also estimated that based on rates from 2005-2007, 40.77% of men and women born today will be diagnosed with cancer of all sites at some time during their lifetime (1). This data suggests that there is a compelling need for improvement in the prevention and management of cancer. Recent research has identified a new perspective on the origin and metabolism of cancer. The invention, on reviewing the research, proposes oral liposomally encapsulated glutathione for the management of cancer may offer significant advances in the prevention and management of cancer. As will be reviewed, the use of glutathione in the management of cancer has been previously discouraged as it has been taught that glutathione will increase the resistance of cancer cells to chemotherapy agents. After reviewing the insights into the metabolism of cancer, the inventor believes that a lack of glutathione and the accompanying oxidative stress may actually increase the likelihood of developing cancer and contribute to the growth of cancer.

The findings from Dr. Michael Lisanti's studies suggest that cancer cells are actually parasites that feed off of the cells around them by turning their support cells known as stromal fibroblast cells into a fuel supply. The glycolysis used by the autophagic tumor stromal cells also present a target for modifying the contribution of these cells to adjacent cancer cells.

Abnormal cell growth is the biological hallmark of cancer. The morbidity of this abnormal growth is related to tumor cell invasion of surrounding as well as distant tissues. In spite of intense research into the origin and perpetration of cancer cells, the field of cancer research remains filled with contradictions and confusion. The prevailing paradigm of the origin of cancer was established 85 years ago by Otto Warburg and has led to a focus on the gene alterations in cancer cells (2). The original research that has been carried into current time shows that cancer cells use fermentation and glycolysis for energy production, with a decreased usage of mitochondrial based oxidation-phosphorylation energy production normally found in cells. The original observations on cancer metabolism remained confusing over the years and led to a theory that gene damage in the nucleus of the cell was responsible for cancer and over time that seemed to confound the confusion about cancer metabolism. One example of this confusion is found in trying to explain how rapidly proliferating cancer cells that have a high energy demand rely on a relatively inefficient method of energy production like glycolysis as is taught currently (3). The need to prevent the continued growth of cancer has led to the use of treatments to remove (surgery) cancer cells, and/or, in combination, kill cancer cells using oxidation producing therapies such as radiation and chemotherapy. Often radiation, drugs or chemotherapy are administered, to “shrink” the tumor followed by resection (surgery), and further radiation, chemotherapy or other drug treatment with serious quality of life issues. The recently discovered new paradigm of cancer metabolism suggests an alternative explanation for cancer cell survival. This new perspective leads to concepts of treating cancer that are dramatically different from the approach that has been in use for decades. Landmark research led by Michael Lisanti, Md., PhD at the Kimmel Cancer Institute is showing that the metabolism of cancer cells may actually be quite different than previously thought and that therapies that increase oxidation of the cancer and surrounding cells should be reconsidered. It is possible that the therapies oriented to increasing oxidation stress may, in some cases, contribute to the perpetuation of the cancer.

The prevailing thinking about the origin and treatment of cancer cells at the present time, 2010, are based on the observations by Warburg in 1927 that cancerous tissue is associated with the utilization of glucose (aerobic glycolysis) and the production of lactic acid (2). Recent research by Lisanti shows that the cancer cells utilize the same type of metabolism as normal cells, i.e., oxidative phosphorylation in the mitochondria. The biochemical pyruvate supplies the electrons and protons (hydrogen ions) that are used in the oxidative phosphorylation pathway to efficiently produce ATP. Lactic acid can also be formed related to the fermentation of glucose. The pyruvate used in normal cells is formed from the tricarboxylic acid cycle also known as the Krebs cycle. Lisanti's work shows that the source for pyruvate and lactic acid used in the oxidative-phosphorylation of cancer cells is supplied by the surrounding support cells, which are known as the tumor stroma. It appears that oxidation stress creates an environment which causes the fibroblasts in the cancer stroma to alter their metabolism from that normally found in cells, in fact to switch form oxidative phosphorylation to that of glycolysis. In summary, the result for the fibroblasts is that they are transformed from cells using normal mitochondrial metabolism to cells using glycolysis, which is a much less efficient method of energy production, but it produces end products such as pyruvate and lactic acid that can be used by the cancer cells. The fibroblast support cells also known as stromal cells, have decreased mitochondrial function and undergo a process called autophagy, in which they “self-digest” components of the cell including the mitochondria. Thus the stromal cells adjacent to cancer cells become cells which produce the biochemical fuel for cancer cells.

### DESCRIPTION OF FIGURES

FIG. 1 shows prostate tissue viewed under a microscope. From Wikipedia, http://en.wikipedia.org/wiki/Stroma_%28animal tissue %29, Dec. 1, 2010.

FIG. 2 shows a fibroblast which are shown as elliptical purple-stained striated objects in center-right area of figure. The view is of a histopathologic image of a gastrointestinal stromal tumor of the stomach from Wikipedia, http://en.wikipedia.org/wiki/Gastrointestinal_stromal_tumor, Dec. 1, 2010.

FIG. 3 shows a fibroblast as a stained object in lower left quadrant of figure in a view of fibroblasts in cell culture. From Wikipedia, http://enwidipedia.org/wiki/Bibroblast, Dec. 1, 2010.

FIG. 4 is labeled: “Arginine is metabolized to nitric oxide (NO) and citrulline under optimal circumstances. In the presence of oxidative stress, some arginine is metabolized to assymetric dimethyl arginine (ADMA). The ADMA inhibits the production of NO from arginine. Oxidative stress inhibits the further breakdown of ADMA to citrulline (thereby increasing the presence of ADMA) and increases the generation of reactive nitrogen species (RNS) from NO. In the presence of adequate reduced glutathione (GSH), NO is metabolized to S-nitrosyl glutathione (GSNO), which has greater stability and a longer half-life than NO, and has vasodilation effects on the endothelium, similar to NO.” The figure is cited from the following: An indirect pathway leading to depletion of NO and related to oxidative stress involves an increase in production of asymmetric dimethylarginine (ADMA), which has been linked to arterial dysfunction.(28) Individuals with arterial dysfunction have been observed to have higher levels of ADMA and lower levels of arginine compared to normotensive individuals.(29) Elevations of cholesterol, oxLDL or tumor necrosis factor (TNF) have been shown to slow the degradation of ADMA, contributing to the increase of circulating ADMA (30,31).

### ENERGY PRODUCTION IN CELLS

Oxidative phosphorylation (oxidative phosphorylation) is a metabolic pathway that occurs inside mitochondria that uses energy released by the oxidation of nutrients to produce adenosine triphosphate (ATP). This pathway is used in mammalian cells apparently because it is a very efficient way to transform the energy of foods to ATP and is much more efficient than the alternative process glycolysis. The oxidation of a single glucose molecule via oxidative phosphorylation in the mitochondria produces 36 ATP's while the glycolytic metabolism of a glucose molecule results in only 2 ATP's per glucose molecule. The use of oxidative phosphorylation in the mitochondria gives a significant energy advantage to cells using this method of energy production.

Biochemical reactions such as glycolysis, the citric acid cycle, and beta oxidation, produce the reduced coenzyme NADH. NADH contains electrons with a high energy potential released when the body uses this energy potential in a step-wise fashion to avoid immediate release of the potential which might disrupt cells, passing the electrons from NADH along a membrane in the mitochondria that contains a series of enzyme complexes (I-IV) that can release a small amount of the electrons energy at each complex. Arrayed along the mitochondrial inner membrane are lipoprotein complexes that can accept the electron, release part of the energy and allow the electron to be released to travel to the next lipoprotein complex in a chain like fashion. The passage of electrons along this chain of complexes is known as the electron transport chain (ETC). After traveling the ETC the electrons are incorporated into a molecule of oxygen, and with the addition of 2 hydrogens (H+) is then turned into water. As oxygen is used in this process oxidative-phosphorylation is also referred to as respiration. In terms of energy production, while an ATP can be formed at each complex in the electron transport chain (ETC), the big payoff in energy production occurs by using the energy of the ETC to pump protons (H⁺) across the inner membrane of the mitochondrial. The protons build up in the intermembrane space of the mitochondria and create an electrochemical gradient across this membrane. The high concentration of protons (low pH) can then push protons through a fifth enzyme embedded in the mitochondrial membrane called ATP synthase. The protons pumped through ATP synthase produce a large number of ATP's. Therefore, the cells using this oxidative phosphorylation process have a distinct advantage over the other forms of energy production such as glycolysis. In cells with functioning mitochondria about 88% of the energy is made by oxidative phosphorylation (4). The remaining 12% of energy is produced by glycolysis in the cytoplasm and through the metabolic cycle known as the Krebs cycle or the tricarboxylic acid cycle (TCA) in the mitochondrial matrix.

Glycolysis refers to the breakdown of glucose with a release of energy for the formation of ATP and also the formation of pyruvate and lactic acid. This process takes place in the watery cytoplasm of the cell, but can provide these materials to the mitochondria. Normal cells use glycolysis in situations where the energy demand of the cell exceeds the supply of oxygen. This occurs in muscle cells during heavy exercise and leads to the use of anaerobic metabolism using glycolysis.

Warburg's initial observations on cancers show that cancer cells utilize glucose and form lactic acid. In cancer cells the use of glycolysis occurs in the presence of oxygen and led to the concept of aerobic glycolysis as a part of the cancer cell metabolism. Subsequent research showed that that mitochondrial oxidative phosphorylation was defective in cancer cells. The combination of this information led to the current prevailing concept that cancer cells have altered DNA that lead to the alterations in their metabolism and uncontrolled growth.

In cancer cells, the acids (high concentration of H⁺, protons, with a resulting low pH) produced by glycolysis in the cytoplasm around mitochondria can also diffuse across the outer mitochondrial membrane. This can add to the supply of protons in the intermembrane space and add additional protons to feed through ATP synthase and increase the production of ATP.

### Liposomally Encapsulated Reduced Glutathione Raises Cell Levels of Glutathione

Recent unpublished studies have documented the surprising ability of the present invention of liposomal glutathione to raise glutathione in tissues to a level higher than that found routinely in cells which the inventor now postulates will be useful in battling cancer. An unpublished study was done in 2010 by B. Lucchesi at the University of Michigan showing this phenomenon. Ischemia (low blood flow with decreased oxygen) followed by reperfusion (return of blood flow) is known to deplete glutathione. In a rabbit in vivo model, after the administration of oral liposomally encapsulated reduced glutathione described in this invention, the level of intracellular glutathione in ischemic/reperfused tissue was almost 30% higher than the animals not fed the oral liposomally encapsulated glutathione. The elevation of glutathione was dose dependent with the animals fed the glutathione for 7 days on a once a day schedule showed higher levels than the animals fed glutathione for only 3 once a day doses. Findings of elevated glutathione in the tissue were also observed using just 3 days of twice a day doses. The dose used in the study was 1 teaspoon of liposomally encapsulated glutathione containing 420 mg of reduced glutathione per teaspoon. The finding of the ability of oral liposomally encapsulated glutathione to be able to maintain glutathione levels higher than untreated tissue documents the absorption into the systemic system as well as the cells of and the tissues of a mammal. This finding supports the use of oral liposomally encapsulated glutathione in mammals that may have hypoxic tissues due to metabolic or vascular perfusion abnormalities.

Another unpublished study in 2010 by V. Venketaraman at Western University investigated the effect of N-acetyl cysteine (NAC) and liposomally encapsulated glutathione to prevent the replication of intracellular Mycobacterium tuberculosis after infecting the cells with the organism. Previous work by Venketaraman has shown that raising glutathione levels with NAC in this cell culture model will limit the growth of Mycobacterium tuberculosis (TB). The study shows that both NAC and liposomally encapsulated glutathione were able to limit the growth of the organisms to a level below 1000 colony forming units per milliliter (CFU/ml). NAC at 10 millimolar reduced the CFU/ml to 8,000, while the liposomally encapsulated glutathione at 5 micromolar concentration reduced the CFU/ml to 6,000 CFU/ml. This data demonstrates that liposomally encapsulated glutathione is over 2000 times more potent than NAC in maintaining the function of macrophages undergoing the oxidative stress of an intracellular infection.

An additional unpublished study shows that liposomally encapsulated reduced glutathione formulated per this invention has a significantly increased absorption and function in the macrophages from individuals with HIV that are undergoing infection with M. tb. The absorption of the liposomally encapsulated glutathione is 1000×'s more efficient than the glutathione precursor N-acetyl cysteine (NAC) in restoring normal glutathione levels and restoring the glutathione related function of slowing the replication of M tb in macrophages taken from individuals with HIV . . . “Glutathione Supplementation Improves Immune Function in HIV+Macrophages,” Morris D, Guerra C, Khurasany M, Guilford T, Venketaraman V, (unpublished, Western University of Health Sciences, Pomona, Calif. 91766, USA) (“Morris D”).

The surprising and novel finding in the unpublished Morris D et al study of the dramatic absorption of liposomally encapsulated reduced glutathione compared to N-acetyl cysteine (“NAC”) explains the ability of this formulated form of liposomally encapsulated reduced glutathione to restore macrophage function back to the M1 function.
“In a previous study we observed elevated levels of TGF-β in both the plasma and macrophage culture supernatants of HIV+ macrophages [42]. This elevated TGF-β will compromise the amount of GCLC present inside the cell; consequently, supplementing the raw materials [such as with NAC] for de novo synthesis in HIV+ individuals who are over expressing TGF-β will not result in the same increased production of reduced GSH that is observed in individuals who are not over expressing TGF-β. In addition, this phenomenon may explain why 1GSH [the liposomally encapsulated reduced glutathione of this invention] at lower concentrations than NAC is more effective at raising the concentration of reduced GSH in HIV+ macrophages than in HIV− macrophages. Supplementing with an 1GSH formulation provides complete GSH molecules to cells, circumventing the enzymatic pathway responsible for GSH production, without the requirement for the cell to construct the tripeptide. This may also explain why treatment with 1GSH seems to raise the ratio of reduced GSH to GSSG at much lower concentrations than NAC, as cells treated with NAC will have to produce new molecules of reduced GSH utilizing their own enzymatic machinery. [emphasis added, citation omitted].” Morris et al at pp. 17-18. (To be published shortly in 2013)

The ability to maintain cell function by raising glutathione directly during an infectious process in the cell is novel and has not been previously reported. The observation that liposomally encapsulated glutathione is 2000 (two-thousand) times more effective in maintaining glutathione and the ability of the cell to limit replication of an intracellular infectious agent such as TB is also novel and previously unreported.

Oxidative stress occurs when there is an excess of molecules containing uncoupled electrons. Molecules with uncoupled electrons are highly reactive and will easily (meaning at low threshold energy levels) “grab” an electron, causing a change in the donor molecule. Oxygen is a strong acceptor of electrons and is used to accept the electrons that have “donated” their energy to produce ATP in the process of oxidative phosphorylation. In the process of oxidative-phosphorylation some radicals of oxygen, that is, oxygen that is still looking for an electron (and its companion hydrogen) are formed. The radicals of oxygen, formed primarily in mitochondria, are known as reactive oxygen species (ROS). One of the most notable is the hydroxyl ion, OH⁻, which not only has a high number of unpaired electrons, but also is ionically negative attracting it strongly to other species to ionically react and form ionic bonds. The “grabbing” of the electron and proton from lipoproteins and enzymes (proteins) causes damage to these materials and interferes with or stops their function.

### The Role of Mitochondria Energy Production in Cancer

The focus on abnormal energy production in mitochondria has led to a debate on the origin of the mitochondrial dysfunction; i.e. is the mitochondrial dysfunction due to gene abnormalities affecting mitochondria or are the gene abnormalities found in cancer due to the abnormalities in mitochondrial function which lead to altered forms of energy production? While this theory was debated over 50 years ago (5), the general view in 2010 is that gene mutations and chromosomal abnormalities underlie most aspects of tumor initiation and progression including the Warburg effect and impaired respiratory function (4). Thus, the gene theory of cancer argues that changes in the DNA of the cell leads to cancer, while the metabolic impairment theory argues that the formation of free radicals in cancer cells affects genes resulting in the wide varieties of DNA alterations seen in cancer. This discussion is more than theoretical as it has been pointed out that if gene mutations are the primary cause of cancer, then the etiology of the disease may be very complicated resulting in the multiple mutations seen in cancers and would require multiple solutions for its management and prevention. On the other hand, if impaired energy metabolism is primarily responsible for cancer, then most cancers can be considered a metabolic disease requiring fewer and less complicated solutions (4).

Lisanti's work begins to clarify some of the confusion about cancer as it suggests that there are two types of cells at play in the tumor mass. The cancer cell itself, while an altered cell, continues to use oxidative-phosphorylation-based-respiration for its energy production. Lisanti has shown that the secondary cells, the fibroblast support cells, have been altered by oxidative stress resulting in impaired mitochondrial function and increased aerobic glycolysis. These altered cells have been shown to be the cells that use aerobic glycolysis and that these cells have decreased mitochondrial function. Damage to the fibroblast support cells has caused these cells to self ingest their mitochondria and to rely on aerobic glycolysis to produce energy. These cells are referred to “autophagic tumor stromal cells”. This theory also explains why such a large number of factors such as radiation, chemicals, viruses, inflammation as well as other provocative agents in the environment contribute to cancer (6). The common factor linking these varied agents is that these provocative agents increase the oxidative stress. Lisanti has also shown the autophagic stromal cell formation, which occurs with oxidative stress and can encourage the formation of cancer cells

The rapid and persistent growth of cancerous tumors led to the use of methods of killing cancer cell populations using methods that generate an excess of free radicals that are able to kill cells. Surgical removal of the tumor mass was one of the first modalities used to treat cancers, but recurrence of the growth both locally and distant from the original site lead to consideration of other methods of eradicating the cancer cells. Radiation was one of the early modalities used in cancer therapy. Chemical agents that had a radiation-like (radiation mimetic effect) oxidizing effect were subsequently developed and became known as chemotherapy agents. In addition, a different approach in the past decade research focused on developing drugs that prevent the formation of blood vessels that bring oxygen to cancer cells in the hopes that these “antiangiogenic” drugs would block the nutrient supply to the cancer. When this theory was first published, it was received with a great flourish including write-ups in major newspapers (7). To date, the angiogenesis theory has not shown decisive benefit in human cancers and cancer treatment remains focused on creating an excess of oxidative stress in cancer cells. The newest research by Lisanti sheds new light on the fundamental mechanisms related to cancer development as well as the origins of the nutrient supply for cancer cells. The inventor concludes, contrary to standard practice, that antiangiogenic drugs and the use of radiation and oxidation producing chemotherapy (“oxidative stress creating modalities”) may aid in physical destruction of cancer cells. However, because these modalities rarely kill all of the cancer cells, and by their nature create oxidative stress, the oxidative stress creating modalities create salutary conditions for the growth of remaining cells. The treatment by oxidative stress creating modalities may effectively preserve and enhance remaining cancer cells, which unfortunately are the strongest cells most able to resist the insult from the oxidative stress creating modality, and further, weaken other cells to facilitate metastasis. This may, in part, explain why there is an anecdotal feeling that when cancer returns, it seems to return “with a vengeance,” and the end of a period of remission often seems to spell doom for the patient. The new research suggests the current treatments to cause oxidative stress appear to be the exact opposite of what is needed to eliminate cancer cells. As we will see, these modalities may actually feed the cancer. As stated, “all” of the cancer is rarely completely eliminated or, as is the case for some cancer cells, the cancer cells may be relatively unaffected by the insults of radiation and oxidation producing chemotherapy. For those cancers refractive to treatment, the radiation and oxidative stress enhance the conditions for the tumor to grow.

The energy producing mechanism used in normal cells called oxidative phosphorylation is very efficient (4) It turns out that cancer cells use the same nutrients that normal cells use for the process of making energy. In normal cells, the Krebs cycle is used to form pyruvate from foods and can form lactate and pyruvate from fermentation and glucose metabolism (glycolysis). The inventor' review of research suggests that cancer cells can also utilize lactate and pyruvate to feed them into the cancer cells oxidative phosphorylation mechanism that produces ATP efficiently. This information is a totally new concept by itself. But the second revolutionary concept is that source of the lactate and pyruvate is from the adjacent cells. It turns out that the cells adjacent to the cancer cells have been damaged by excess oxidation and have undergone a process in which they autodigest their own components including their own mitochondria and are forced to use glycolysis to produce energy. The auto-digest process is known as autophagy, which means “self eating”. The process of digesting the mitochondria is known as mitophagy.

This process of autophagy results in a decrease in mitochondria and adaptation of the cell energy process to produce lactate and pyruvate that will then be taken up and used by cancer cells. The support cells called fibroblasts are very susceptible to this process. Fibroblast cells make up the support structure for epithelial cells and are called stroma. These observations have led Lisanti to name his theory the “Autophagic Tumor Stroma Model of Cancer (8).

Both normal tissue and cancer cells are supported by cells called fibroblasts. It is thought that cancer arises when the well differentiated cells, which are usually epithelial cells, transform into a more primitive form and do not follow the usual rules of controlled tissue growth. A biopsy or excision of cancer tissues will reveal both cancer cells and also the support cells called stromal cells.

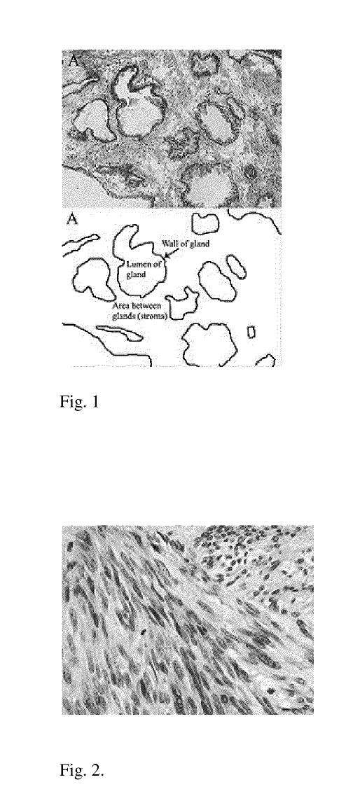

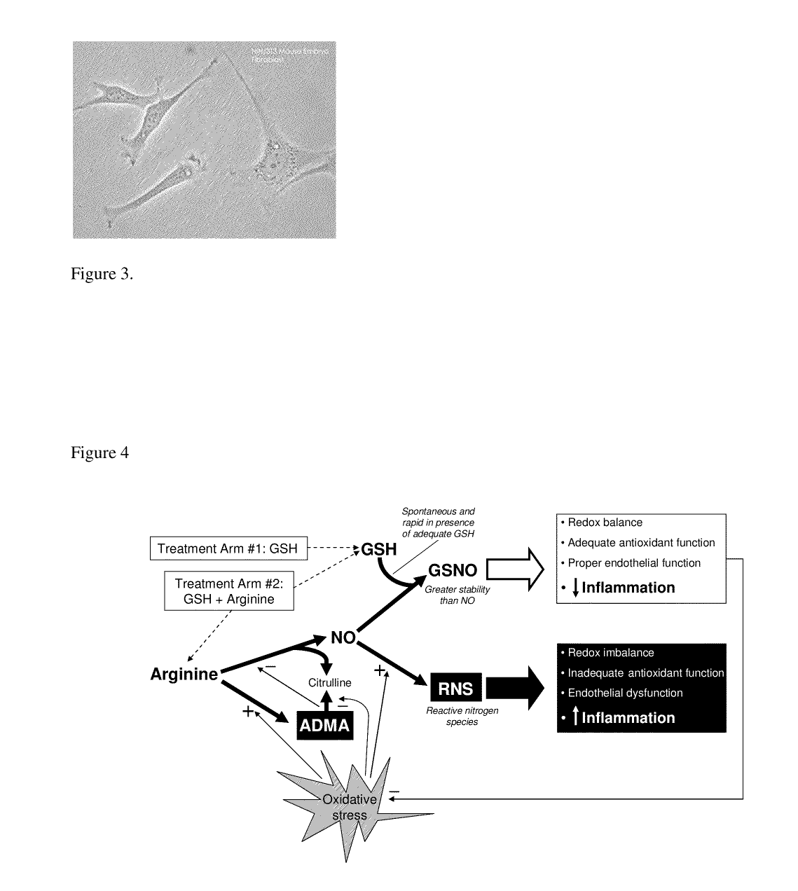

Cancer associated fibroblasts comprise a majority of the cells found in tumor stroma (9). Stroma refers to the non-functional supportive frame work for support of a tissue. For example in a glandular tissue such as prostate, the stroma fills the space between the gland tissue. FIGS. 1,2, and 3 illustrate the relative position.

The autophagy response in the fibroblast stromal cells alters both their metabolism and their appearance and changes them into a nutrient source for the cancer cells. This alteration causes the stromal cells to undergo the “Warburg Effect” and to become the fuel source for cancer. The revolutionary concept demonstrated by Lisanti is that it is not the cancer cells that undergo the “Warburg effect”, but it is the surrounding stromal cells that undergo the “Warburg effect.” Meanwhile the cancer cells maintain their “normal” method of energy production and also enjoy an increased nutrient supply that bolsters their metabolism. Lisanti describes this situation as the “Reverse Warburg Effect”.

When normal cells encounter severe oxidative stress and poor oxygen supply they will undergo a self-destructive process called apoptosis, which is defined as a self destruction of the cell with an autodigestive process that is followed by the cells' removal by scavenger cells. In the situation of the autophagic tumor stromal cells, oxidatively damaged fibroblasts undergo only part of the process, with the self digestion involving the mitochondria. The presence of the nutrient and fuel source from the stromal cells has also been shown to make the cancer cells more resistant to oxidative stress and apoptosis. The metabolic parasitic relation between cancer cells and the supply of nutrients from the oxidatively stressed autophagic fibroblasts offers a new concept of cancers and an opportunity for new anti-cancer therapies.

It has been known for some time that cancer cells have adequate glutathione and that the peripheral blood of patients with cancer is low in glutathione and has increased oxidative stress (10). In spite of this observation, the use of methods to raise glutathione has been discouraged because of concern that boosting glutathione would increase the resistance of cancer cells against oxidative therapies like radiation or chemotherapy (11). Thus, the medical literature has taught away from the use of antioxidants and other methods to raise glutathione in individuals with cancer. The utilization of glutathione in individuals with cancer has previously been discouraged as it has been shown that resistance of the tumor cells to oxidative therapies has been correlated with higher glutathione levels (35) (36). It now appears that the systemic increase in oxidative stress is an extension to the whole body of the local oxidative phenomenon that causes the stromal cells to be damaged by oxidation and to become the source of fuel for the cancer. The systemic oxidation stress associated with cancer is often associated with loss of tissue mass called wasting. The process of weight loss and wasting that is associated with cancer is also referred to as cachexia. It now appears that the peripheral oxidation and cachexia associated with cancer is related to the cancer process slowly turning the whole body into a fuel source for the cancer cells.

Agents that interfere with glycolysis are referenced as methods of reducing the contribution of autophagic cells to the production of pyruvate. These agents include SB-204990, 2-deoxy-D-glucose (2DG), 3-bromopyruvate (3-BrPA, Bromopyruvic acid, or bromopyruvate), 3-BrOP, 5-thioglucose and dichloroacetic (DCA) (12). DCA is a mimetic of pyruvate which interferes with pyruvate dehydrogenase kinases (PDK1-4), causing a decrease in glycolysis and shifts the use of pyruvate back to oxidation in the mitochondria. Importantly, it has been shown that DCA does not have a direct cancer cell action. The mechanism of action on the autophagic tumor stromal cells has not been previously referenced (13). The dose for dichloroacetic acid can range from 10 mg/kg to 100 mg/kg with 35 mg/kg a preferred dosage (14). The dose may be 10 mg/kg, 20 mg/kg, 30 mg/kg, 40 mg/kg, 50 mg/kg, 60 mg/kg, 70 mg/kg, 80 mg/kg, 90 mg/kg, or 100 mg/kg. Stimulating cell metabolism with thyroid medication such as triiodothyronine (cytomel) 5 micrograms to 15 micrograms with or without sustained release formulation or with caffeine is also suggested as this will increase the metabolism and glycolysis in the autophagic stromal cells and will allow a lower dose of dichloroacetic acid or other glycolysis-disrupting (antiglycolytic) agents to have a greater effect on the autophagic stromal cells. The use of the glycolysis disrupting agents alone or in combination with caffeine or triiodothyronine is referenced as methods with or without the simultaneous use of liposomally encapsulated reduced glutathione. While these agents have been discussed as anticancer agents in previous literature (14) (13) there is no reference noting their use in the modulation of the autophagic fibroblasts found adjacent to cancer cells. There has been no previous literature referencing the use of glutathione in combination with antiglycolytic agents, a combination referenced in the current application. Additionally, there has been no reference for the use of dichloroacetic acid or similar agent to decrease glycolysis in the autophagic stromal cells. The encapsulation of glycolytic agents in liposomally encapsulated formulation is also referenced as method of increasing the delivery of the antiglycolytic agent to the tumor. The liposome encapsulation may be done with either the lecithin liposome or the self forming liposome as described in the later examples. The preferred method is encapsulation of dichloroacetic acid in liposomes ranging in size from between 20 nm and 10 microns at a concentration of 400 mg per 5 cc of liposomal liquid.

The inventor believes that transformation of the autophagic cells by oxidative stress can also be slowed or prevented using agents such as vitamin E succinate (D-Alpha-tocopherol succinate), which helps maintain the function of mitochondria. The succinate form is water-soluble and can encapsulate in the liposome formulation of the present invention. The vitamin E succinate can feed succinic acid to complex II of the four complexes involved in oxidative phosphorylation in the mitochondria. Complex I is compromised early in oxidative stress situations and the loss of the ability to use pyruvate for oxidative phosphorylation will speed the loss of mitochondrial function with subsequent autophagy and mitophagy of the cell. The encapsulation of D-Alpha-tocopherol succinate in liposomally encapsulated formulation is also referenced as method of increasing the delivery of the D-Alpha-tocopherol succinate to the autophagic tumor stromal cells as well as the cancer cells. The liposome encapsulation may be done with either the lecithin liposome or the self forming liposome as described in the example. The preferred method for all of the liposomal formulations in the invention, including for D-Alpha-tocopherol succinate, is encapsulation in liposomes ranging in size from between 20 nm and 10 microns at a concentration of 400 mg of D-Alpha-tocopherol succinate per 5 cc of liposomal liquid.

The purpose of the present application is to reference the use of liposomally encapsulated reduced glutathione as method of treating the oxidation stress that occurs in stromal cells adjacent to cancer cells as a means of preventing and reversing the formation of an “autophagic tumor stroma” (15)) has shown that the use of N-Acetyl Cysteine (NAC), a building block of glutathione can have the effect of reversing the oxidative stress in the stromal cells. However as explained below, NAC requires energy to formulate intracellular glutathione, which energy is often not available in a cell with a compromised energy function. Cysteine, as found in NAC has been the only possible oral method, however inefficient, to increase glutathione though it is not particularly effective and no showing has been made of in vivo application. Liposomally encapsulated reduced glutathione, the present invention, has been shown in an unpublished study (Lucchesi) to raise glutathione levels in tissues after oral ingestion in a rabbit model of ischemia (low oxygen) followed by the return of blood flow and oxygen (i.e., reperfusion) injury. Low oxygen has been shown to increase oxidative stress and will increase the possibility of forming the autophagic tumor stroma. Supplying adequate glutathione in accord with the present invention can prevent the formation of the altered stromal cells which has the effect of stopping the fuel supply for the cancer. Diminishing the fuel supply for the cancer cells will result in the death of the cancer cells. The novelty of the current application lies in the surprising attributes of liposomally encapsulated glutathione according to this invention to deliver glutathione internally to tumor stromal cells in a manner that exceeds the ability of plain glutathione to deliver glutathione to tumor stromal cells.

In addition to diminishing the fuel supply, the invention protects macrophage cells essential to immune surveillance and tumorcidal activity. There has been ongoing debate regarding the question of whether the immune system positively or negatively controls neoplastic progression The importance of maintaining macrophage function in regard to cancer resistance is that the inflammation associated with cancer sends cytokine signals which invite macrophage migration to cancer tissue, such as breast cancer. In breast tumors, macrophages constitute up to 35% of the infiltrating inflammatory cells (16). The role of immune function and cancer may be considered as three roles, cancer immunosurveillance, equilibrium, and escape (17). Macrophage cells are phagocytic cells that patrol in tissues and play a significant role in immune defense against cancer as cells presenting tumor-associated antigens to tumor-infiltrating lymphocytes and as cytotoxic effector cells. However, macrophages have been shown to have a dual role in regard to tumor growth, (18). While macrophages participate in surveillance and immune eradication of tumors on one hand, it has been shown that they can also contribute to tumor growth and the supply of new blood vessels on the other. The response of macrophage cells to tumor is dependent on the type of macrophage that accumulates in the tumor environment.

The activation state of macrophages has two classes. The M1 macrophage produces large amounts of proinflammatory cytokines and is involved in killing pathogens and tumor cells. The M2 macrophage moderates the inflammatory response and promotes new blood vessel formation (angiogenesis) and tissue remodeling. Macrophages found in the tumors are called tumor associated macrophages (TAM). Macrophages in tumors that are growing appear to be of the M2 variety. The tumor-cidal activity of macrophages results from the release of reactive oxygen species and nitric oxide (NO), which can kill tumors (18).

In addition, macrophages release tumor necrosis factor-alpha, which, as its name implies kills tumor cells (18). It has also been shown that S-nitroso-L-glutathione, has a direct killing action on tumor cells (19). S-nitroso-L-glutathione is formed from the combination of NO and glutathione. Thus, the lack of glutathione will impair the tumor-cidal function of the macrophage at several levels.

Supplementation with oral L-arginine increases the formation of NO in both animals and humans. (20) The production of NO is dependent on availability of arginine and the function of endothelial derived nitric oxide synthase (eNOS). Asymmetric dimethyl arginine (ADMA) is another product of arginine metabolism. It prevents the normal function of eNOS to both produce NO in what appears to be a competitive fashion.(21) It has been observed that the ratio of available arginine to ADMA is critical in determining the formation of NO and that small changes in this ratio, such as increasing the level of arginine, will overcome the competitive inhibition of ADMA and increase NO production. (FIG. 1). (20,22).

The availability of NO is decreased by oxidative stress by both direct and indirect pathways. In a direct pathway, the presence of oxygen or superoxide, converts NO to reactive nitrogen species such as nitrogen dioxide and peroxynitrite.(23-26) Exposure to peroxynitrite, other reactive nitrogen species, and reactive oxygen species, results in decreased NO availability.(27) An indirect pathway leading to depletion of NO and related to oxidative stress involves an increase in production of asymmetric dimethylarginine (ADMA), which has been linked to arterial dysfunction.(28) Individuals with arterial dysfunction have been observed to have higher levels of ADMA and lower levels of arginine compared to normotensive individuals.(29) Elevations of cholesterol, oxLDL or tumor necrosis factor (TNF) have been shown to slow the degradation of ADMA, contributing to the increase of circulating ADMA (30,31).

In the presence of oxidation stress, macrophage metabolism of arginine will favor the function of arginase, which produces ornithine as opposed to nitric oxide synthase, which produces nitric oxide. Thus, at least one of the tumor-cidal mechanisms will be diminished in macrophages with depleted glutathione. In addition macrophages that make ornithine, which is a precursor of polyamines needed for tumor cell replication and are associated with tumor progression (32). It is the ability of macrophages to produce nitric oxide or ornithine that determines if the macrophage type is M1 (nitric oxide producing) or M2 (ornithine producing) (33). While Lisanti has shown that raising glutathione with the use of NAC may inhibit the formation of autophagic tumor stromal cells (34) there has been no publication of the use of glutathione either by the precursor NAC or with the use of liposomally encapsulated glutathione to support macrophage cells to maintain the production of nitric oxide from arginine as a method to maintaining the tumor-cidal action of macrophages. The combination of arginine plus glutathione is also referenced for use as prophylaxis for prevention of cancer, prophylaxis against metastasis of cancer as well as for the treatment of cancer. The preferred method is the use of liposomally encapsulated glutathione 2 teaspoons combined with arginine 1000 mg taken orally twice a day on an empty stomach. Another method of cancer treatment is the encapsulation of GSNO (19). S-nitroso-L-glutathione) in either the lecithin or the self forming liposomes of the current invention for use as an anti-cancer therapy. Liposomal encapsulated GSNO, molecular weight 336.3, is 80 mg/ml of liposomal GSNO and the dosing is ½ teaspoon to 4 teaspoons orally twice a day which range includes ½ teaspoon, 1 teaspoon (5 ml), and ½ teaspoon increments up to 4 teaspoons orally twice a day.

An article by Zeevalk (37) shows delivery of glutathione in experimental dishes to brain cells. Unpublished data shows an extraordinary increased ability of the invention to supply glutathione to macrophage cells whose function is implicated in cancer disease as explained momentarily. Unpublished data described above by Venketaraman, 2010 shows that liposomally encapsulated glutathione can maintain the function of macrophage cells even after they have been infected with an intracellular bacteria that is known to compromise the function of these cells and to cause the death of the macrophage cells absent administration of the particular liposome with reduced glutathione. The study shows that function of macrophage cells infected with the intracellular organism Mycobacterium tuberculosis can be supported 2000 (two thousand) times more efficiently with the addition of liposomally encapsulated glutathione to the cell culture than with NAC. The surprising increased potency of liposomally encapsulated glutathione in maintaining macrophage function is the basis for referencing that liposomally encapsulated glutathione according to this invention can be a novel treatment for the management of cancer. Specifically, liposomally encapsulated glutathione is referenced for the ability to support macrophage and natural killer cell function, critical components of the innate immune system, which is altered by oxidative stress in cancer (3). Additional advantages of the use of liposomally encapsulated glutathione compared to NAC are found in the observation that liposomally encapsulated reduced glutathione provides glutathione directly to cells and by passes the need for construction of glutathione by energy dependent pathways in the cell. It has been documented that under conditions of severe infection that NAC is not adequate to raise glutathione (38). Additionally, it has also been shown that NAC will not raise glutathione during the oxidative stress that accompanies poorly controlled type 1 diabetes in adolescents (39). Thus a method of supporting glutathione directly in the microenvironment of the tumor is needed to maintain the tumor-cidal activities of macrophage, as well as to prevent the oxidative stress in the cancer microenvironment that leads to the formation of autophagic tumor stromal cells.

Macrophage cells play an important role as a major component of the innate immune system. The innate immune system uses preprogrammed information to recognize cells that are foreign such as invading bacteria or transformed normal cells and uses the process of phagocytosis to engulf, kill, analyze and remove foreign material or invaders. In tissues, macrophages also do the day to day work of cleaning up and removing cells that have become defective or aged and have undergone a process of self removal called programmed cell death (PCD). PCD generally occurs when there is a disruption of the cell machinery, especially in regard to the formation of energy from mitochondria or a change in the components of the cell membrane. Failure of the macrophage system will allow a buildup of the abnormal, partially dying cells. The process of engulfing cells undergoing programmed cell death is also a function of macrophages such as the removal of compromised neutrophils. The process of engulfing and “digesting” dying cells is a complex process and interruption of this function can result in the perpetuation of infectious cells inside the macrophage. This process is displayed in macrophages undergoing infection with tuberculosis, for example. Maintaining adequate glutathione in the macrophage is critical for this function. While it has been reported that NAC can provide antioxidant support for macrophages undergoing infection with Tb, this application references the surprising and unexpected ability of liposomally encapsulated glutathione to support the function of macrophage cells infected with Tb with a potency 2000 times greater than NAC. (Venketaraman 2010, unpublished) It does not follow that merely because NAC can support Tb infected macrophages, that therefore, liposomally encapsulated glutathione produced according to the modes of this invention would yield a surprising and unexpected 2000-fold increase in advantage. The absorption and the effect of liposomally encapsulated reduced glutathione is unexpectedly significantly better than NAC. For instance NAC is less effective and does not raise glutathione for treatment of poorly controlled Type I diabetes and sepsis. The novel observation underlying this invention is that the energy necessary to produce glutathione from cysteine is often not present in cells suffering insult or with impaired energy function which is why cysteine or NAC are significantly less effective. The advantage that the liposome encapsulated glutathione brings in supporting macrophage cells is significant and represents a significant advantage in being able to supply glutathione to infected phagocytic cells by providing glutathione intact directly to the cells. Supplying the glutathione directly to the cell has a distinct advantage because it avoids the need to build glutathione from the constituent building blocks cysteine, glutamine and glycine. These 3 amino acids are capable of being made by the body, however cysteine is generally the component that is the least available and is therefore known as the rate limiting factor in the production of glutathione. The production of glutathione from cysteine or NAC requires energy. For example, it is estimated that 5 ATP's are needed to support glutathione production. Plain glutathione is not imported directly into the cell, so it is broken down into the component amino acids by peptidases found on the outside of the cell. Then the components of the glutathione are imported into the cell, where 3 ATP's are used in the 2 step process of forming glutathione.

Cells undergoing infection or oxidative stress have a decreased amount of energy available and may not be able to convert the building blocks of glutathione such as cysteine into the complete glutathione compound. This leaves the normal cell receiving an insult at a distinct disadvantage as the specific substrate glutathione is needed to support the function of glutathione peroxidase and remove free radicals such as peroxide. Despite the fact that NAC would require too much energy, and the cell is at an energy disadvantage, and the dearth of positive results with respect to use of oral glutathione to treat cancer, which use of oral glutathione had been criticized, the inventor forged ahead in spite of the research and found unique properties useful in treating cancer by a unique liposomal encapsulation of glutathione according to the present invention.

The presence of an increased amount of free radicals in the cell and mitochondria will perpetuate the formation of the autophagic stromal cells. Liposomally encapsulated glutathione has a 100 times potency in restoring glutathione than plain glutathione to astrocytes depleted of glutathione (37). The previously unreported finding that liposomally encapsulated reduced glutathione has a potency 2000 times higher than NAC in supporting macrophages undergoing infection is a positive unexpected surprising effect. It suggests a unique ability that is not available with cysteine or plain glutathione, and makes the current invention, stabilized and encapsulated liposomal reduced glutathione particularly effective for supporting macrophages undergoing inflammatory stress. (Venketaraman 2010, unpublished)

The ability to maintain macrophage function with the present invention, liposomally encapsulated reduced glutathione is important as there may be additional mechanisms at play in the local cell environment which increase oxidative stress and can interfere with macrophage function. The role of metals and mycotoxins, two very different toxins, which are removed by macrophages are discussed below. A buildup in macrophages of one or both of these toxins can lead to an excess of oxidative stress in the macrophage that compromises its function in a manner that allows the macrophage to become tumor supportive instead of tumor killing. Thus the present invention, liposomally encapsulated glutathione, also offers a unique and novel method of supporting macrophage function in the oxidized environment associated with the accumulation of metals or the ingestion of invading mycological invaders that may contain mycotoxins. Metals have been implicated in cancer causation (40).

The ability of liposomally encapsulated glutathione to increase the level of glutathione in normal tissues above a normal level has not been previously reported. In addition it has been taught that reduced glutathione is maintained in a narrow range of about 10:1 over the amount of oxidized glutathione in the cell, so it has been thought that it is unlikely that a “supra normal” level of glutathione can be achieved in the cell. Previous work by Zeevalk monitoring cell levels in vitro of glutathione has shown that liposomally encapsulated glutathione can restore levels of glutathione back to the expected levels of normal cells, but there was no indication that supra-normal levels that would protect cells from the effects of hypoxia would be found (37).

The present invention is administration of liposomally encapsulated reduced glutathione as a method for the maintenance of oxidatively stressed macrophage and NK cells that has the ability to maintain infected macrophage cells with a potency 2000 times that of NAC. This has not been previously reported or suggested. This ability may be enhanced by the use of hydroxylated lecithin in the formation of the liposomes. It has been shown that in oxidative conditions that macrophages display a receptor CD36 that increases the absorption of oxidized lipoprotein like oxLDL. It is likely that the hydroxylation used in the formation of the liposomes of the present invention increases the absorption of liposomally encapsulated glutathione in the macrophages and may contribute to the extraordinary and unexpected benefit seen with this invention.

In regard to the weight loss associated with cancer which phenomenon is called cachexia, a biochemical was initially associated with cachexia and had such a high correlation that it was initially called cachectin. It was subsequently discovered that another biochemical that was elevated in cancers and seemed to have a cancer limiting effect which became identified as tumor necrosis factor-α. Over time research showed the two differently named entities were the same biochemical and it became known as tumor necrosis factor-α (TNF-α). TNF-α has been shown to be a cytokine associated with inflammation and is released as part of the cytokine storm in influenza, for example. Paradoxically, it has been shown that when TNF-α is elevated in individuals with persisting cancer it is associated with a poor prognosis in cancers such as breast cancer (41). This may be related to the observation that IL6 or TNF-α; also significantly reduced plasma membrane associated caveolin-1 (“Cav-1”) (42), the absence of which are characteristic of autophagic stromal cells. Inflammation mediated by cytokines such as tumor necrosis factor-α (TNF-α) increase oxidative stress in cells. While this may have a tumorcidal effect on the cancer cells, it can also cause the formation of autophagic tumor stromal cells. TNF-α functions by increasing oxidative stress by depleting glutathione (43). Cancer cells without the fuel supply to make energy to maintain a constant production of glutathione n the cancer cell will be more susceptible to TNF-α

Cachexia due to cancer is a complex metabolic disorder, including loss of adipose tissue due to lipolysis, loss of skeletal muscle mass, elevation of resting energy consumption, anorexia, and reduction of oral food intake (44). The mechanism is thought to involve a down regulation of the energy utilization through a chain of events which includes compromise of arginine metabolism resulting in changes in endogenous nitric oxide synthase production (eNOS), decreased mitochondrial function with a decrease in mitochondrial biogenesis and a decrease in fat metabolism at the mitochondrial level. Cancer cells require large amounts of glucose to grow and can use as much as 5 times the amount of glucose as normal cells (45). The cancer cell's continued need for glucose keeps the host individual in a constant state of gluconeogenesis in the liver. As the amount of glucose being used exceeds the oxygen in growing tumors lactic acid will be formed (46) in addition to pyruvate. The release of lactic acid stimulates the liver to make glucose via the enzyme glucose via the enzyme phosphoenol pyruvate carboxykinase. As more glucose is made, more lactic acid is made and this creates an energy draining cycle called the Cori cycle that is found to be present in individuals with cancer related cachexia. In individuals with cancer the increased level of TNF-α combined with additional cytokines such as IL-1 and IL-6 add to the progression of cachexia (47). This invention proposes to curtail the adverse effects of that energy-draining process.

The elevation of oxLDL and CRP in the blood of individuals with cancer suggests a method of monitoring cancer using these biochemicals as biomarkers. 0xLDL is both a biomarker and pathogenic factor involved in the compromise of macrophage metabolism. The oxidative stress and autophagy in stromal cells also creates stresses on the normal epithelial cells that can result in mutations that go on to cause previously normal cells to change to cancer cells. While it has been thought that these oxidative changes occur due to low oxygen, it is likely that any cause of oxidative stress can trigger this sequence. Metals considered toxic such as mercury and lead have been shown to be present in breast cancer (40). It seems likely possible that the oxidative damage in the fibroblasts in these tissues will trigger the autophagic response as well as the sequence of events that move on to form cancer. It has been shown that the oxidative and autophagy-altered stroma will cause the DNA of the adjacent epithelial cells to become cancer cells (48).

Creating oxidation stress in stromal cells causes these stromal cells to stop production of a protein called caveolin-1 that cells use to carry nutrients into the cell. Caveolin protein is found in flask shaped structures called caveoli that are part of the membrane of cells. Caveolin lines the caveoli which are used in molecular transport, cell adhesion and signal transduction. Caveoli play a prominent role in ingesting and removing various materials including lipids such as cholesterol and are particularly abundant in endothelial cells (49). Cholesterol imported into cells via caveoli are carried directly to intracellular sites where cholesterol is metabolized for use in the cell (23). Caveolin is involved with both the import and export of cholesterol and functions in a way similar to the way that plasma lipoproteins move lipids between tissues (50). Cells undergoing oxidative stress and autophagy lose the formation of caveolin-1 early in the damage cycle. Dr. Lisanti has shown that a lessening of the caveolin-1 biomarker in the stromal cells indicates an increased risk of growth and spread of the cancer. Lisanti MP, Martinez-Outschoorn UE, Chiavarina B, Pavlides S, Whitaker-Menezes D, Tsirigos A, et al. Understanding the “lethal” drivers of tumor-stroma co-evolution: Emerging role(s) for hypoxia, oxidative stress and autophagy/mitophagy in the tumor micro-environment. Cancer biology & therapy. 2010; 10(6). Cited in PubMed; 20861671 http://www.landesbioscience.com/jounals/cbt/article/13370/./. This interaction has been shown to be present in breast and prostate cancers and it is likely that it will be present in other cancers also.

As noted, the loss of caveolin distinguishes the autophagic tumor stromal cells. At the same time, caveolin-1 is increased in the cancer cell itself (51). It has been shown that a high level of intracellular cav-1 expression in cancer tissue is associated with metastatic progression of human prostate cancer (52) (51) and other malignancies, including lung, (53) renal (54) and esophageal squamous cell cancers (55)

At the same time that the amount of caveolin is disappearing in the oxidatively stressed stromal cells, the increased lipid metabolism of the cancer cells results in an increase in Cav-1 protein in cancer cells and even in the levels of Cav-1 in the serum of men with prostate cancers compared to men with benign prostatic hyperplasia (56). Cav-1 can also be elevated in patients with elevated risk of cancer recurrence after radical prostatectomy surgery (20) and has been shown to be overexpressed in various malignancies, including cancer of the colon kidney, bladder, lung, pancreas, ovary, and in some types of breast cancer (51). An important component of studies using immunostains to identify caveolin on biopsy tissues is that the caveolin is higher in the cancer cells than surrounding cells and that cav-1 immunostaining is expressed only in a relatively small percentage of prostate cancer cells. Cav-1 has also been shown to occur in metastatic cells (57).

The importance of Cav-1 in the stromal cells relates to the lethality of tumors. It has been shown that in triple-negative breast cancers (referring to estrogen receptors, progesterone receptors, and HER-2 receptors), patients with high stromal Cav-1 have a 75.5% survival rate at 12 years, which can be contrasted to patients with an absence of Cav-1 who have a survival rate of less than 10% at 5 years post-diagnosis (15).

The increase in glycolysis in the tissue compromised by cancer, including the surrounding tissue, as described by Warburg (39) has led to the conclusion that there is an impairment of the mitochondria as well as an increase in glycolysis (3). However, the impairment is not in the cancer cells themselves, but in the mitochondrial function of the fibroblast stroma cells surrounding the actual tumor cells. This dysfunction results in the autophagy of the surrounding fibroblast stroma cells including the mitochondria, leaving the surrounding fibroblast stroma cell with only aerobic fermentation for energy production, thereby generating enhanced amounts of products for the cancer cell. It has been thought that the repression of mitochondria affords the cancer cell with a cell-death resistant phenotype making them prone to malignant growth (58). Lisanti's work indicates that the supply of fuel from the autophagic support cells is the reason that cancer cells become resistant to cell death. The inventor postulates that the extra supply of fuel allows the cancer cells to not only upregulate the defenses of the cancer cell against oxidative stress such as increasing the genes for glutathione S-transferase to improve detoxification, but also to have the ATP energy needed to form more of the substrate, reduced glutathione, needed to maintain an increased activity and function of the enzyme glutathione-S-transferase. Tumor resistance to chemotherapy agents such as cisplatin has been associated with the presence of glutathione (11). In the past there has been a generally accepted proscription against the use of antioxidants such as glutathione or glutathione enhancing agents because it has been thought that this type of activity would enhance the cancer cells ability to protect itself against radiation or chemotherapy agents (59). As described in this application, this inventor takes a different theory of cancer function and proposes to stabilize cells by engaging in prevention of autophagy using stabilized and encapsulated liposomal reduced glutathione, and in the preferred mode, in higher glutathione concentrations encapsulated in the liposome described in this application, which liposome can be formulated to be consumed orally. It is turning out that the lack of antioxidants actually increases the autophagic changes in the cells surrounding the cancer cells and results in an increases the chances of the cancer cells surviving.

An unpublished study, Morris D, Guerra C, Khurasany M, Guilford F, Saviola B, Huang Y, et al. Glutathione Supplementation Improves Macrophage Functions in HIV. JOURNAL OF INTERFERON & CYTOKINE RESEARCH. 2013, suggested and supported by the inventor suggests that the invention can be utilized to improve macrophage function in HIV. The inventor analogizes from this that the research shows that the invention will have a surprising result as to cancer.

Similarly, the inventor commissioned research at the University of Michigan also as yet unpublished showing the surprising effect of the invention in reversing and controlling oxidative stress which supports the inventor's theory behind this invention as to cancer. Lauver et al, University of Michigan Medical School, “Oral Pretreatment With Liposomal Glutathione Attenuates Reperfusion Injury in Rabbit Isolated Hearts,” to be published in the Journal of Cardiovascular Pharmacology (2013), That study shows that contrary to the usual degradation in the gut, the invention, purchased from Your Energy Systems, LLC of Palo Alto, Calif., in the amount of approximately 428.8 mg of GSH administered in 5 ml doses, had the following abstracted result:
“A liposomal preparation of glutathione (lipGSH) capable of oral administration was investigated for its ability to attenuate tissue injury and increase myocardial glutathione levels in an isolated heart model of reperfusion injury. Male, New Zealand white rabbits were assigned randomly among four groups: control and daily oral administration of lipGSH for three, seven or fourteen days. At completion of the dosing regimen, hearts were harvested and perfused in a retrograde manner with the use of a Langendorff apparatus. The hearts were subjected to 30 min of global ischemia followed by 60 min of reperfusion. Hearts from lipGSH-treated rabbits exhibited better recovery of left ventricular contractile function during reperfusion and had attenuated oxidative damage. Furthermore, hearts from lipGSH-treated animals had increased myocardial tissue levels of GSH demonstrating effective absorption of lipGSH.”

The invention proposes that based on the Lauver et al unpublished research, the administration of liposomally encapsulated glutathione pursuant to the invention would raise the level of intracellular glutathione by at least 30%, particularly in tissues oxidatively stressed or otherwise stressed by cancer.

The present invention proposes the combination of serum levels of oxLDL, HDL, CRP and Cav-1 as a novel combined collection of biomarkers that can be used to the progression and risk of progression of cancer as well as offering a means of monitoring the response to therapy for cancer. The level of oxLDL <45 U/L U is normal with levels >63 U/L elevated. Individuals with prostate cancer were monitored and it was observed that individuals with caveolin scores<0.13 ng/mL had low risk of recurrence, while individuals with >0.13 ng/mL had an increasing risk of recurrence (60). C-reactive protein levels using the high-sensitivity CRP test in serum are normally below 1 mg/l. Levels of 2.5 are associated with increased risk. Thus a combination of scores in the ranges set forth below would indicate either no cancer, or a low risk cancer while abnormal outside these levels would suggest increasing risk of recurrence or an aggressive cancer or both.

It is proposed that a surprising effect of the use of EDTA and glutathione by intravenous infusion or oral liposomal encapsulation of the glutathione for ingestion will be the reduction in the formation of the autophagic stromal cells. There is no previous literature suggesting these materials be used as single agents or in combination for the purpose of preventing or reversing the formation of autophagic stromal cells. Lisanti does suggest that raising glutathione will help reverse the progressive loss of Cav-1, autophagy and mitophagy (34) (15) and references an article by Gao 2007, which also mentions the use of N-acetyl cysteine and other antioxidants as anti-cancer agents. However, each of these articles references these materials as a method of reducing hypoxia and the decrease in release of hypoxia inducing factor. The concept of the use of EDTA and reduced glutathione encapsulated in liposomes to remove both toxic metals and metals such as iron which can be found normally in cells, especially in the mitochondria where iron is a component of the enzyme complexes associated with oxidative phosphorylation, decrease the oxidative stress factors that are inducing the formation of autophagic tumor stroma is novel to the current application. The dose for liposomally encapsulated calcium ethylene diamine tetraacetic acid (“caEDTA”) or disodium ethylene diamine tetraacetic acid (“EDTA”) is 100 mg to 3 grams in a single dose. The preferred dosing schedule is calcium ethylene diamine tetraacetic acid (“caEDTA”) 500 mg every other day for 3 weeks and then reassess. The dose for liposomally encapsulated glutathione is 1 teaspoon containing 430 mg reduced glutathione using a dose of ½ to 4 teaspoons per day. The preferred dose of liposomally encapsulated glutathione for individuals with cancer is 2 teaspoons twice a day. The dose can be 100 mg, 200 mg, 300 mg, 400 mg, 500 mg, 600 mg, and so on by 100 mg increments up to 3000 mg which is 3 grams.

It has been observed that there are similarities between yeast and mammalian cells in response to impaired respiration. It has been proposed that early carcinogenesis often occurs in a low oxygen environment and the ability to metabolize glucose anaerobically would be an advantage for cancer cells (61). A protein, hypoxia-inducible factor-1α (HIF-1α) is produced in response to low oxygen levels in mammalian cells. Normally, HIF-1 α is rapidly broken down in the cell, however, in prolonged low oxygen states it can become a stable protein. HIF-1α plays a critical role in cell survival during low oxygen as HIF-1α induces expression of pyruvate dehydrogenase kinase 1 and most major genes involved with glucose uptake, glycolysis, and lactic acid production [127]. It has been found that HIF-1α is elevated in most cancer cells. The mechanism being the formation of HIF-1α in cells with normal oxygen levels remains unresolved although it has been shown that certain viruses, such as the hepatitis B virus can affect mitochondria and stabilize HIF-1α. It is interesting in this regard that carcinogenesis, whether arising from viral infection or from chemical agent, produces similar impairment in respiratory enzyme activity and mitochondrial function (4).

In addition to virus, chemicals, and metals it has also been observed in previously unreported data from RealTime Lab, Dallas Tex., using either monoclonal or polyclonal antibodies specific to selected mycotoxins it has been shown that mycotoxins such as ochratoxin, and trichothecene are frequently found in tumor tissue. It has been known for some time that aflatoxin (aflatoxin B1) is associated with cancer (62). These toxins cause damage to cells by increasing oxidation stress. As macrophages ingest the molds and their mycotoxins these cells are susceptible to compromise by the mycotoxin. The oxidative impairment of macrophages will have several results that increase the susceptibility to cancer. Oxidatively burdened macrophages are less able to move normally and the specialized macrophages known as dendritic cells or antigen presenting cells will not be able to migrate to the local lymph nodes and thus, no secondary or T cell mediated antibodies will be made. These cells will also be inefficient in processing the antigenic material as the oxidative stress will prevent the adequate function of the killing process, which uses oxidative stress in specialize subcellular compartments to kill, digest and prepare ingested (phagocytized) organisms. It is possible that compromised macrophage cells that have ingested fungal invaders and the macrophage has become compromised by the mycotoxins and develop a fusion of the macrophage and tumor cells (63) (64), which has been documented for macrophage and fungal cells. The perpetuation of the production of mycotoxin in a compromised macrophage explains the finding of mycotoxin in cancer tissue. Macrophages make up a large portion of the inflammatory infiltrate in most, if not all cancer types (65) and contribute up to 80% of the total tumor mass, depending on cell type (especially brain tumors) (66). In a small series of brain lesions specimens, it has been shown that of 13 lesions diagnosed as astrocytoma samples tested for mycotoxin, 5 of 7 contained tissue consistent with tumor tissue and were positive for one of both of aflatoxin or ochratoxin type mycotoxins. The remaining 6 samples should only inflammation yet 4 of the 6 contained one or both mycotoxins. In breast tumor tissue, 3/3 tumors and 1 lymph node from a breast tumor positive individual were positive for the mycotoxin Ochratoxin A. Various lung cancers including mesothelioma (ochratoxin), adenocarcinoma (aflatoxin) and bronchial alveolar carcinoma (aflatoxin and ochratoxin) have been shown to contain mycotoxin. In evaluation of renal cell carcinoma, 3 of 3 tumors were shown to contain the mycotoxin ochratoxin A (OTA) (67).

Mycotoxins have been shown to increase oxidative stress in cells. Ochratoxin A exposure can increase oxidation stress directly, but has also been shown to down-regulate the genes related to the formation of enzymes that facilitate the connection of glutathione to toxins, called glutathione-S-transferases (GST's) (68). It is also conjectured that there may be a generalized decrease in cell antioxidant protection after exposure to Ochratoxin A (69). No publications suggest the use of glutathione for the management of ochratoxin A related cancers.

Aflatoxin is known to be associated with cancer, especially hepatocellular carcinoma. Aflatoxin B1 has been shown to impair phagocytosis and intracellular killing (70). However, the administration of plain (non liposomal) reduced glutathione orally in mice which were pretreated with aflatoxin B1 showed no benefit in two studies in terms of decreasing the incidence or size of hepatic nodules (71) or tumors (72). This finding teaches away from the expectation of benefit to be observed with by simply placing glutathione into liposome.

Studies also suggest that aflatoxin and ochratoxin may produce a synergistic toxicity (73, 74).

Whether the origin of mycotoxin in tumor tissue is from infection with a fungal nidus, fusion of fungus and macrophage or the persistence of an intracellular infection with fungus, the production of mycotoxin produces an oxidizing agent that may cause oxidation stress in local cells and may trigger the autophagic phenomenon in the surrounding cells. The previously described surprising potency of the present invention, liposomally encapsulated reduced glutathione, stabilized to inhibit conversion to GSSG, in maintaining macrophage function compared to other methods of raising macrophage glutathione such as NAC is referenced as a method of maintaining macrophage function after exposure to mycotoxins.

It has been postulated that macrophage cells have many similarities to metastatic cancer cells (75). While virus is mentioned in the publication as a potential element capable of compromising mitochondrial function, there is no mention of the glutathione status, the role that metals play in inducing oxidative stress, nor is the role of mycotoxins mentioned as possible factors contributing to the decreased tumor killing function of macrophages or the possibility that these materials could lead to a state of confusion in the macrophage cell which facilitate its conversion to a cancer cell. A recent review of the use of liposomes in the treatment of cancer mentions the desirability of diverting macrophages from an inefficient state in terms of tumorcidal effects, a condition described as M2 macrophages to a more efficient macrophage form, termed M1 in which the tumorcidal and tumor rejection properties are improved. However, the study is clear in stating that there is no current method of effecting this change. Accordingly, there is no mention of glutathione in liposomes in the review (76), as this invention proposes. The present invention provides reduced glutathione in a liposome that is ingested into macrophages at a surprising rate that is 2000 (two-thousand) times more efficient than NAC in terms of preserving macrophage function during an infectious process. See, Venketoraman, described above. There is also no mention n the review of liposome based therapies for cancer for the use of liposomally encapsulated glutathione to maintaining glutathione in the macrophage cells to increase the ability to metabolize arginine to nitric oxide, and thus maintain the M1 type macrophage function. Thus, the hydroxylated lecithin liposomes containing glutathione in its reduced stated described in this application are referenced as a surprisingly more efficient method of supplying glutathione to macrophages in general in the system as well as those associated with cancers and tumors.

In order to maintain the supply of arginine available to macrophages during the stress of interacting with an invading organism or the stress of inflammation related to the response to tumor tissue the concomitant use of exogenous arginine together with liposomally encapsulated glutathione is referenced in this application. The arginine can be included in the liposome or separately, taken orally at the time of ingesting the LRG. The preferred dose of arginine for the maintenance of M1 macrophages is 1000 mg in conjunction with liposomally encapsulated glutathione 2 teaspoons, each teaspoon containing 420 mg reduced glutathione taken orally twice a day on an empty stomach. The dose of arginine may range from 500 mg in an adult to 2000 mg at each dose, while the dose of liposomally encapsulated glutathione according to the invention in the adult may range from 1 teaspoon per day to 8 teaspoons per day and be 1 to 8 teaspoons per day in 0.5 teaspoon increments, in one or more doses.

Additional combinations of antifungal agents plus liposomally encapsulated glutathione are also referenced as methods of management of cancer including the combination of statin drugs, which have antifungal activity.

Antifungal medications include:

Polyene antifungals. These are not absorbed orally:

Amphotericin, Nystatin, Griseofulvin, Flucytosine, Terbinafine, Caspofungin.

Statins have also been shown to have antifungal qualities, possibly due to the ability of statins to inhibit production of isoprenylated proteins that are essential to fungi (77).

Statins include: Simvastatin, fluvastatin, lovastatin, atorvastatin, rosuvastatin, pravastatin.

Imidazoles are taken orally:

Miconazole—(Miconazole nitrate), Ketoconazole, Clotrimazole—marketed as Lotrimin or Lotrimin AF (and Canesten in the UK). Econazole, Bifonazole, Butoconazole, Fenticonazole, Isoconazole, Oxiconazole, Sertaconazole—marketed as Ertaczo in North America. Sulconazole, Tioconazole

Triazoles are taken orally:

Fluconazole, Itraconazole, Isavuconazole, Ketoconazole, Ravuconazole, Posaconazole, Voriconazole, Terconazole. Including New triazole antifungal agents having C6S7 or S6C7 bridges as disclosed by Wu, Nian in a U.S. Patent Application published as 20100143455.

Allylamines

Allylamines inhibit the enzyme squalene epoxidase, another enzyme required for ergosterol synthesis:

Terbinafine—marketed as “Lamisil” in North America, Australia, the UK, Germany and the Netherlands. Amorolfine, Naftifine—marketed as “Naftin” in North America.

Butenafine—marketed as Lotrimin Ultra.

Echinocandins

Echinocandins inhibit the synthesis of glucan in the cell wall, probably via the enzyme 1,3-β glucan synthase:

Anidulafungin, Caspofungin, Micafungin

Other

Ciclopirox—(ciclopirox olamine), Tolnaftate—fungicidal, marketed as Tinactin, Desenex, Aftate, as well as other names. Undecylenic acid—organic unsaturated fatty acid derived from natural castor oil, fungistatic as well as anti-bacterial and anti-viral. Flucytosine, or 5-fluorocytosine, is an antimetabolite.

Griseofulvin—binds to polymerized microtubules and inhibits fungal mitosis.

Haloprogin—discontinued due to the emergence of more modern antifungals with fewer side effects

The preferred combination is 200 mg of itraconazole orally per day for 2 to 16 weeks in combination with oral liposomally encapsulated reduced glutathione 422 mg (1 teaspoon) twice a day.

Higher doses of oral liposomally encapsulated reduced glutathione are also referenced with doses up to 4 ounces a day in divided doses may be integrated into the therapy protocol.

The preferred combination is 200 mg of voriconazole orally per day for 2 to 16 weeks in combination with oral liposomally encapsulated reduced glutathione 422 mg (1 teaspoon) twice a day. Higher doses of oral liposomally encapsulated reduced glutathione are also referenced with doses up to 4 ounces a day in divided doses may be integrated into the therapy protocol.

Intranasal antifungal therapy in the form of irrigation or topical intranasal spray may also be used in combination with oral liposomally encapsulated reduced glutathione. The objective of this therapy is to reduce the presence and growth of fungal material in the nose and adjacent sinuses.

The dose of oral liposomally encapsulated reduced glutathione is oral liposomally encapsulated reduced glutathione 422 mg (1 teaspoon) twice a day.

The preferred therapeutic for intranasal therapy is
0.3% (3 mg/mL) amphotericin B suspension in a nasal spray twice a day (total volume 800 μl) in each nostril, twice a day, during 4-16 weeks.
Another preferred method for the nasal spray is to use 100 mg of fluconazole in 500 ml of normal saline solution administered as 5 sprays (0.5 cc/spray) in each nostril twice daily.
Another preferred method for the nasal spray is to use Itraconazole 0.1% Nasal Spray 5 sprays each nostril twice a day.

The intranasal antifungal therapy should be accompanied by doses of oral liposomally encapsulated reduced glutathione in a range of 1 teaspoon twice a day to 4 ounces a day in divided doses may be integrated into the therapy protocol.

Plain glutathione used orally is not an option for this therapy as plain glutathione is not absorbed after oral ingestion in humans (78). A rat study of the removal of a radio-tagged metal (CO-60) from the liver, performed at Pacific Northwest National Laboratory with oral liposomally encapsulated reduced glutathione confirms this observation. The tissue from the control animals (water) served as the 100% of the toxin remaining in the tissue. The animals receiving:
a. Control (water only) showed 100% of the toxin remained=0% removal
b. Plain glutathione, oral, in water showed 90% of the toxin remained=10% removal.
c. Intravenous glutathione showed 30% of the toxin remaining=70% removal.
d. Liposomal reduced glutathione showed 40% of the toxin remaining=60% removal.

The data from this study is consistent with the observation that liposomally encapsulated glutathione is almost as effective as intravenous glutathione in removing the toxin. The plain glutathione has little if any absorption or efficacy.

Oral liposomally encapsulated reduced glutathione that is uniquely designed to be absorbed a) across the mucosa of the nose, mouth, gastrointestinal tract, b) after topical application for transdermal, or c) by intravenous infusion of with or without liposome encapsulation is prepared under the method and according to the composition described as follows:

As metals are known to increase the oxidative stress in cells and tissues, a third component of cancer management is referenced as the use of metal chelators such as EDTA, DMPS or DMSA in addition to the use of the LRG and an antifungal agent.

### Strategies Using the Tumor Stromal Cell Demand for Glucose to Increase Absorption of Therapeutic Agents

As the autophagic stromal cells import increased amounts of glucose, an additional therapy to reduce the number of autophagic stromal can be accomplished by a technique that takes advantage of the use of insulin to potentiate a form of cancer treatment known as Insulin Potentiated Therapy (ITP) to increase the absorption of chemotherapy agents in combination with the administration of reduced glutathione intravenously or encapsulated in liposomes (liposomal glutathione) is also a novel therapy for the treatment and prevention of the formation of the autophagic fibroblasts associated with cancer cells. The use of insulin given intravenously prior to the infusion of chemotherapy agents which can be given at lower doses and thus will have less “collateral” damage on normal cells has been described for the treatment of cancer. This technique is described in a paper by Lasalvia-Prisco in 2004, in which the combination of insulin followed by methotrexate is described as producing a significant antitumor response not seen by methotrexate or insulin alone in individuals with breast cancer. The technique has not been accepted by mainstream medicine and has been derided as unproven, thus teaching away from the use of IPT (See AM Cancer Society website referenced below as well as the Quackwatch website, Why You Should Stay Away from Insulin Potentiation Therapy). There are no subsequent articles describing the use of this technique.

At the same time, the use of glutathione enhancing agents during cancer treatment has also been previously discouraged due to the fact that cancer cells with increased glutathione may have increased resistance to chemotherapy agents such as cisplatinum, melphalan and doxorubicin (79) (80). In fact, it has been taught that depletion of glutathione with an inhibitor of glutathione synthesis may increase the sensitivity of cancer cells to these chemotherapy drugs (79). Enzymes such as glutathione S-transferase that facilitate the interaction of glutathione with toxins as well as chemotherapy agents, have been shown to be highly active in cancer cells. The ability of cells to produce glutathione requires a continuing supply of ATP. The capacity of cancer cells to maintain elevated intracellular levels of reduced glutathione (GSH) is likely due to the increased availability of lactate and pyruvate fuel from the autophagic tumor stromal cells. The ability to continue to make high levels of glutathione and maintain cell the ATP dependent proteins that remove toxins and chemotherapy agents from the cell called multidrug resistant proteins. Multidrug resistance proteins such as MDR1 and MRP1 act as pumps which can remove chemotherapy drugs and lower the intracellular drug concentrations of agents used in chemotherapy (81) (82). The high level of biochemical fuel provided by the autophagic cancer cells for the mitochondria of cancer cells allows the production of adequate ATP to overcome chemotherapy drugs and increases the resistance of cancer cells to chemotherapy agents. Thus, the combination of the ability to make ATP and the presence of increased expression of multidrug resistance proteins will increase the chemotherapy resistance of cancer cells. The ability to eliminate the fuel source for cancers, the autophagic tumor stromal cells increases the efficacy of chemotherapy agents. So in spite of articles that teach that increasing glutathione will increase the resistance of tumor cells, the use of the present invention, oral liposomally encapsulated glutathione will actually increase the efficacy of chemotherapy agents as it reduces the ability of cancer cells to produce the energy needed to maintain the multidrug resistant protein pumps. The doses of oral liposomal reduced glutathione to achieve these results are daily doses ranging from 3 mg/kg to 100 mg/kg of glutathione but most preferably 6 mg/kg to 36 mg/kg as found in the preferred liposomally encapsulated glutathione preparation which contains 82 mg liposomally encapsulated glutathione per milliliter.

In radiation, or in chemotherapy which functions as a radiation mimic in terms of causing oxidative stress and generating free radicals, there is evidence of systemic oxidation in the blood of cancer-afflicted patients reflecting there is damage to peritumor cells and tissue. The invention functions to bring the peritumor cells to normal redox balance by supplying the liposomally encapsulated reduced glutathione intracellularly.

The use of ITP+ glutathione (IV or as oral liposomally encapsulated glutathione) represents the combination of two approaches that have literature suggesting teaching away from their use. The American Cancer Society has a web page (83) which describes concerns about the therapy and has no positive statements regarding this approach. The use of glutathione in the management of cancer has been discouraged in research articles for decades describing methods to lower glutathione in cancer cells to enhance the efficacy of radiation therapy (84) and chemotherapy (79). It is proposed in this application that the use of liposomally encapsulated glutathione and ITP as either single therapies or in the preferred mode as a combination therapy offers a management of cancer that fits the newly described physiology of cancer and has not been previously proposed. The general concept of insulin potentiation therapy is that because cancer cells and autophagic cells are using glucose metabolism, there are a relatively larger number of glucose intake sites in those cells. In the presence of insulin, the presence of more glucose intake sites selectively enables more penetration of chemotherapeutic agents into cancerous and autophagic cells with more glucose intake sites over normal cells. Thus, relatively more cancerous and autophagic cells are killed. The liposomally encapsulated reduced glutathione cooperates with the just referenced therapy by protecting normal cells and limiting degeneration of macrophage function, thereby avoiding enhancement of energy to the cancer cells by diminishing the creation of compromised normal cells.

Chemotherapy agents with which the present invention is intended include, but is not limited to:
Alkylating agents such as cisplatin, carboplatin, oxaliplatin, Busulfan, Cyclophoshamide and Melphalan
Antimetabolites such as azathioprine, mercaptopurine, pyrimidine, 5-Fluorouracil, and Fludarabine, and antifolates such as Methotrexate, pralatrexate and pemetrexed.
Vinca alkaloids such as Vincristine, Vinblastine, Vinorelbine, Vindesine
Antitumor Antibiotics such as Bleomycin, Doxorubicin and Idarubicin
Mitotic Inhibitors including Taxanes such as paclitaxel, Docetaxel, Etoposide and Vinorelbine
Cyclophosphamide (Cytoxan, Neosar)
Salinomycin

The usual cisplatin dose for the treatment of metastatic ovarian tumors as an example of high dose therapy in combination with cyclophosphamide is 75 to 100 mg/m² IV per cycle once every four weeks (DAY 1) (m in the units: “m²” referring to height of the patient).

The dose of cyclophosphamide when used in combination with cisplatin is 600 mg/m² IV once every four weeks (DAY 1).

For directions for the administration of cyclophosphamide, refer to the cyclophosphamide package insert.

In combination therapy, cisplatin and cyclophosphamide are administered sequentially.

As a single agent, cisplatin should be administered at a dose of 100 mg/m² IV per cycle once every four weeks.

With the insulin potentiated therapy, the dosing is reduced as low as 1/10^{th} the high dosing and thus would be in combination with cyclophosphamide is 7.5 to 10.0 mg/m² IV per cycle once every four weeks (DAY 1).

The dose of cyclophosphamide when used in combination with cisplatin is 60.0 mg/m² IV once every four weeks (DAY 1).

For directions for the administration of cyclophosphamide, refer to the cyclophosphamide package insert.

In combination therapy, cisplatin and cyclophosphamide are administered sequentially.

As a single agent, cisplatin should be administered at a dose of 10.0 mg/m² IV per cycle once every four weeks.

Bleomycin is a radiation mimic in that it produces an increase in free radicals in cells and damages cell DNA, which is thought to be the mechanism of action of bleomycin toxicity. The activity of bleomycin is dependent on a metal ion cofactor. For example, the bleomycin-Cu(ii) complex will decrease glutathione peroxidase significantly, while free bleomycin decreases the enzyme only slightly (85). Bleomycin also complexes with iron (Fe). The bleomycin-Fe(II) complex is oxygen sensitive which becomes oxidized to Fe(III), yielding an oxygen free radical the is capable of causing oxidative stress damage to the DNA of the cell. The details of this process are reviewed in an article by Chattopadhyay (86). It turns out that in the presence of reducing agents such as dithiothreitol, the bleomycin-Fe(III) complex is cycled back to Fe-(II) allowing for repeated, multiple free radical formation from a single Bleomycin molecule.

The ability of glutathione to perpetuate the toxicity of bleomycin is demonstrated in the article by Chattopadhyay, in which the DNA damaging effect known as clastogenic activity of bleomycin was found to be enhance in the presence of increased intracellular glutathione (86). It was previously observed that in mutant lymphocytes in vitro the administration of bleomycin (BLM) in the presence of glutathione results in a potentiation of the cytotoxic activity of BLM. This potentiation was attributed to GSH acting as a reducing agent in reactivating oxidized BLM (87).

A preferred embodiment is the use of liposomally encapsulated glutathione in conjunction with a formulation of liposomally encapsulated bleomycin. The standard dose of bleomycin is by intravenous infusion weekly or twice weekly: 10-20 U/m² (U referring to mg). For the insulin potentiated therapy bleomycin weekly or twice weekly: 1.0-2.0 U/m²

Another preferred dosage method involves the use of liposomally encapsulated bleomycin either combined with liposomally encapsulated glutathione or given contemporaneously The dosage may also be given orally in a liposome, using either the lecithin based liposome or the Q-some described in the present application. The dose with the lower dose insulin potentiated therapy (ITP) will be bleomycin weekly or twice weekly: 1.0-2.0 U/m².

It has been observed in unpublished research (Hunter, Ohio State University, 2009) that, cells with high glutathione levels will be preferentially damaged by bleomycin. Liposomally encapsulated glutathione can be utilized as a form of therapy will be useful in the treatment of cancer that is resistant to chemotherapy agents. Bleomycin is detoxified in cells using the bleomycin hydrolase enzyme, which is dependent on cysteine. Bleomycin therapy can be accompanied by damage to tissues such as fibrosis of lung tissue, especially in doses over 400 mg. Thus cysteine can be administered as a method of facilitating the metabolism of bleomycin by supplying the substrate for the enzyme bleomycine hydrolase. Cysteine may be supplied as either cysteine or N-acetyl cysteine 500 mg to 2000 mg orally three times a day as a rescue for the normal cells from the toxicity of bleomycin.

Additionally, the cancer cells have higher levels of glutathione relative to the surrounding cells, which are oxidized and have low glutathione. A method of management capitalizing on this observation is to use liposomes encapsulating bleomycin. The activity of bleomycin is enhanced by the presence of glutathione (86). Bleomycin is removed by a hydrolase enzyme called bleomycin hydrolase that requires cysteine for function (88). The administration of bleomycin intravenously in standard doses will affect the glutathione laden cancer cells with a higher degree of efficacy than the surrounding cells. The effect of bleomycin can then be diminished by n-acetyl cysteine (NAC) intravenously, orally, or in liposomes. The cysteine from NAC or plain cysteine will then activate the bleomycin hydrolase, break down the bleomycin after it has had its cell killing activity in the cancer cells, but lessen its effects on normal cells. In summary, the administration of bleomycin intravenously in followed by NAC. The standard dose of bleomycin is by intravenous infusion weekly or twice weekly: 10-20 U/m² For the insulin potentiated therapy bleomycin weekly or twice weekly: 1.0-2.0 U/m² The standard dose of cysteine to follow either form of bleomycin therapy is N-acetyl cysteine in doses from 1 gram to 4 grams per day. A preferred dose following bleomycin therapy is 1200-mg intravenous bolus and 1200 mg orally twice daily for the 48 hours

While it has been known for some time that cancer tissues that are resistant to some forms of chemotherapy may have an enhanced response to bleomycin (89), no specific therapy that takes advantage of the presence of glutathione in cancer cells has been recommended. Additional advantage and a preferred mode of therapy is to use liposome encapsulated bleomycin as many forms of liposome material is absorbed into cancer sites preferentially. For this process the use of the Qusome liposome, described herein, which has been shown to accumulate in tumor tissue is preferred. The QuSome self-forming liposome is of such as size and the presence of the steric stability with PEG results in long circulation and an increased accumulation in the fine trabecular mesh of blood vessels supplying growing tumors. This characteristic of accumulating in the trabecular mesh of blood vessels leading to tumors leads to an improved therapeutic. The accumulation of QuSome self-forming liposomes in the blood vessel supply to tumors will concentrate the dose of bleomycin in this area, so relatively low doses which are non-toxic to other tissues may be utilized.

No publication claims the use of a preparation of a cell permeable preparation of glutathione in a liposomally encapsulated preparation that is capable of oral ingestion for treatment of cancer or cancer metastasis. The goal is to prevent the superoxide (O2-.) accumulation that is usually transformed into a trigger for apoptosis and autophagy by oxidative damage of macromolecules, membranes, and DNA usually indirectly through the generation of more toxic (reactive) radicals such as peroxynitrite (ONOO.—) and hydroxyl radical (.OH) (Martindale and Holbrook, 2002). Superoxide(O2⁻.), acting directly or through the cascade of more toxic molecules can efficiently induce the collapse of the mitochondrial membrane potential, which is abbreviated ΔΨm. The collapse of the mitochondrial membrane potential results in the release of cytochrome c and activation of caspase cascades (Madesh et al., 2005; Stefanec, 2000) which normally results in cell death., but in the case of tumor stromal cells may result in the mitophagy and autophagy, which results in the formation of the aerobic and anaerobic glycolysis associated with autophagic tumor stromal cells.

Additionally, it is likely that contrary to previous teaching, tumor cells are not seeking a less oxidized environment, but prefer a more oxidized environment. The more oxidized a tissue or cellular environment becomes, the more the demand for glutathione production to compensate for the oxidizing agents will be. At some point, cells will succumb to this constant demand and their mitochondria will become damaged by the oxidative stress and to the cell will convert to an autophagic cells. Thus the environment of the tissue will determine the increased risk for metastasis. The more oxidized the tissue environment, the more conducive to the growth of metastatic tumor cells the tissue will become. Thus, while it has been taught that metastasis of cancer cells is part of an attempt to escape oxidative stress, (90) the presence of oxidative-stress-induced autophagic cells producing lactate and pyruvate will actually create an attractive environment or “soil” that will support metastatic cancer cells. The present invention is proposed as a method of lessening the likelihood of metastasis of cancer. The surprising level of glutathione that can be achieved using the oral liposomally encapsulated glutathione will create a tissue environment that is less likely to support cancer growth.

The addition of alkalinization of the microenvironment of the tumor may also be of benefit in compromising the energy source of cancers. The glycolysis and release of acids from the autophagic tumor stromal cells will create an acid (low pH) environment. The H⁺ (protons) from in the microenvironment can enter the cancer cells and pass through the outer mitochondrial membrane. The additional protons will then be trapped in the intermembrane space, which does not release the protons, and will add to the proton pressure supplying the ATP synthetase enzyme, known as the proton pump. The additional protons will add to the production of ATP by the proton pump. Adding alkalinizing materials will reduce the concentration of protons, increasing the pH. The ingestion of liposomes containing alkalinizing material such as bicarbonate will lessen the acidity in the environment of the tumor. The preparation is made by adding sodium bicarbonate solution 1.5% w/w to 8.5% w/w to the formula for the liposomally encapsulated glutathione to make a combination product. An alternative preferred method is to use 1.5% w/w to 8.5% w/w sodium bicarbonate solution by itself, replacing the liposomally encapsulated glutathione in the formulation such the sum of the percentage of glutathione in liposomes w/w plus the sum of the percentage of sodium bicarbonate w/w is 8.25% w/w or 8.5% w/w. This will allow for separate dosing of the liposomal alkalinizing solution. The preparation may be used in the lecithin based oral liposome formulation or in the self forming liposome preparation. Thus the percentage of sodium bicarbonate can be 1.5% w/w, 2% w/w and so on in increments of 0.5% up to about 5.0% w/w of sodium bicarbonate. In the combined composition there would then be, for example, a w/w percentage of 5.0% sodium bicarbonate and 3.5% liposomally encapsulated glutathione, or vice versa, 3% sodium bicarbonate and 5.5% liposomally encapsulated glutathione.

It has also been observed that toxins from molds common to the environment are often associated with tumor tissue and may have compromised the physiology of macrophages in such a way that they have become reservoirs for the mold or fungal metabolism. In effect, the macrophage may become transformed to caveolin positive cancer cells. The physiology related to the formation of autophagic stromal cells can be monitored with a combination of biomarkers that monitor serum levels of caveolin-1, C-reactive protein and oxidized LDL cholesterol.

Deionized water can be used to bring w/w percentages up to 100% in any of the tables or formulations below.

### Dosing

Selenium should also be administered 200 mg per day.

Liposomally encapsulated reduced glutathione (also referred to as liposomal glutathione or liposomal reduced glutathione or liposome-encapsulated glutathione): The preferred dosing schedule of the invention for the treatment of symptoms related to treatment of cancer is 800 mg (2 teaspoons) of the invention to be taken twice a day on an empty stomach (that is do not ingest until 30 minutes after eating solid food)

1 teaspoon of the invention of oral liposomally encapsulated reduced glutathione reduced contains approximately 420 mg GSH.

A preferred mode sets a suggested dose based on body weight. Recommended amounts are for use in the treatment of cancer. For best results it is suggested that the invention be used if there is a finding of cancer. These doses may also be used if there is a finding of an elevation of oxLDL or CRP or other non-invasive indicator of cancer such as a elevation of a cancer marker such as the prostate specific antigen also known as the PSA.

Gently stir liposomally encapsulated reduced glutathione into the liquid of your choice.

### Determine Individual Dose by Body Weight: For Children

Under 30 lbs: ¼-½ teaspoon=100-200 mg GSH

30-60 lbs: ½-1 teaspoon=210-420 mg GSH

60-90 lbs: ¾-1.5 teaspoon=316 mg-630 GSH

90-120 lbs: 1-2 teaspoon=422-844 mg GSH

120-150 lbs: 1½-3 teaspoon=630-1260 mg GSH

Over 150 lbs: 1½-3 teaspoons=630-1260 mg GSH

The invention should be used on a continuous basis.

Children—should use a dose of liposomally encapsulated reduced glutathione equivalent to 60 mg/Kg of body weight daily in divided doses.

These doses should be continued for the duration of the duration of the illness and for purposes of maintaining adequate glutathione in tissues before, during and after therapy for cancer.

### DETAILED DESCRIPTION OF THE PREFERRED EMBODIMENTS

The methods of manufacture described in Keller et al U.S. Pat. No. 5,891,465, U.S. Pat. No. 6,610,322, and U.S. Pat. No. 6,726,924 and U.S. provisional application No. 60/597,041 by this inventor are adopted herein and into the modes of this invention and can be applied to the examples without undue experimentation. Liposomal formulations preferred in this invention can be purchased from Biozone, Inc. of Pittsburgh, Calif. Reduced glutathione can be b purchased from Sigma-Aldrich of St. Louis, Mo. or from Kyowa Hakko USA, Inc., 767 3^{rd} Ave. No. 9, of New York City, N.Y. 10017 with a Western regional office at 85 Enterprise, Suite 430, Aliso Viejo, Calif. 92656. Liposomally encapsulated reduced glutathione can be purchased from Your Energy Systems, LLC, 555 Bryant St., Suite 305, Palo Alto, Calif. 94301.

### Example 1

Liposomal glutathione Drink or Spray 2500 mg per ounce or form suitable for encapsulation or gel

| | % w/w |
|---|---|
| Deionized Water | 74.4 |
| Glycerin | 15.00 |
| Lecithin | 1.50 |
| Potassium Sorbate | 0.10 |
| (optional spoilage retardant) | |
| Glutathione (reduced) | 8.25 |

A lipid mixture having components lecithin, and glycerin were commingled in a large volume flask and set aside for compounding. Hydroxylated lecithin is the preferred ingredient.

In a separate beaker, a water mixture having water, glycerin, glutathione were mixed and heated to, but not more than, 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of liposomally encapsulated glutathione. The amount of glutathione added to the formulation may range from 3.3% to 8.5% or higher. The methods of manufacture described in Keller et al U.S. Pat. No. 5,891,465, U.S. Pat. No. 6,958,160 and U.S. Pat. No. 7,150,883 and U.S. provisional application No. 60/597,041 are incorporated in this description. Concentrations of liposomally encapsulated glutathione from 3.3%, 4%, 5%, 6%, 7%, 7.5%, 8%, 8.5% or 9% liposomally encapsulated glutathione may be formed and utilized for dosing by decreasing the amounts of glutathione and preplacing the material with an increase in the sterile water concentration.

### Example 1A

Liposomally encapsulated reduced glutathione Drink or Spray 2500 mg Per Ounce or Form Suitable for Encapsulation or Gel: In %, according to w/w: Deionized Water 75, Glycerin 15.00, Lecithin 1.50, Extract Potassium 0.10 Sorbate Glutathione 8.5 (reduced)

A lipid mixture having components lecithin, ethyl alcohol and glycerin were commingled in a large volume flask and set aside for compounding. Hydroxylated lecithin is the preferred ingredient.

In a separate beaker, a water mixture having water, glycerin, glutathione were mixed and heated, but not more than, 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. A spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of liposomally encapsulated glutathione. The amount of glutathione added to the formulation may range from 3.3% to 8.5% or higher. The methods of manufacture described in Keller et al U.S. Pat. No. 5,891,465, U.S. Pat. No. 6,958,160 and U.S. Pat. No. 7,150,883 and U.S. provisional application No. 60/597,041 are incorporated in this description.

Concentrations of liposomally encapsulated glutathione from 3.3%, 4%, 5%, 6%, 7%, 7.5%, 8%, 8.5% or 9% liposomally encapsulated glutathione may be formed and utilized for dosing by decreasing the amounts of glutathione and preplacing the material with an increase in the sterile water concentration.

### Example 2

Embodiment two of the invention includes the incorporation of the fluid liposome (such as that prepared in Example 1A) into a gelatin based capsule to improve the stability, provide a convenient dosage form, and assist in sustained release characteristics of the liposome. The present embodiment relates to the use of glutathione in the reduced state encapsulated into liposomes or formulated as a preliposome formulation and then put into a capsule. The capsule can be a soft gel capsule capable of tolerating a certain amount of water, a two-piece capsule capable of tolerating a certain amount of water or a two-piece capsule where the liposomes are preformed then dehydrated.

The liposome-capsule unit containing biologically encapsulated material can be taken in addition to orally, used for topical unit-of-use application, or other routes of application such as intra-ocular, intranasal, rectal, or vaginal.

The composition of examples 1 and 2 may be utilized in the encapsulated embodiment of this invention.

Gelatin capsules have a lower tolerance to water on their interior and exterior. The usual water tolerance for a soft gel capsule is 10% on the interior. The concentration of water in a liposome formulation can range from 60-90% water. An essential component of the present invention is the formulation of a liposome with a relatively small amount of water, in the range of 5-10%. By making the liposome in a low aqueous system, the liposome is able to encapsulate the biologically active material and the exposure of water to the inside lining of the capsule is limited. The concentration of water should not exceed that of the tolerance of the capsule for which it is intended. The preferred capsule for this invention is one that can tolerate water in the 15-20% range.

The methods described by Keller et al, U.S. Pat. No. 6,726,924 are incorporated in this description.

Components are commingled and liposomes are made using the injection method (Lasic, D., Liposomes, Elsevier, 88-90, 1993). When liposome mixture cooled down 0.7 ml was drawn into a 1 ml insulin syringe and injected into the open-end of a soft gelatin capsule then sealed with tweezers. Filling of gel caps on a large scale is best with the rotary die method or others such as the Norton capsule machine.

### Example 3

Liposomally encapsulated S-Nitroso-L-glutathione (GSNO) Drink or Spray 2500 mg per ounce or form suitable for encapsulation or gel

| | % w/w |
|---|---|
| Deionized Water | 74.4 |
| Glycerin | 15.00 |
| Lecithin | 1.50 |
| Potassium Sorbate | 0.10 |
| (optional spoilage retardant) | |
| GSNO | 8.25 |

A lipid mixture having components lecithin, and glycerin were commingled in a large volume flask and set aside for compounding.

In a separate beaker, a water mixture having water, glycerin, glutathione were mixed and heated to, but not more than, 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stifling plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of liposomally encapsulated glutathione. The amount of glutathione added to the formulation may range from 3.3% to 8.5% or higher The methods of manufacture described in Keller et al U.S. Pat. No. 5,891,465 are incorporated into this description or as described before may be used.

### Example 4

Embodiment number four of the present invention includes the creation of liposome suspension using a self-forming, thermodynamically stable liposomes formed upon the adding of a diacylglycerol-PEG lipid to an aqueous solution when the lipid has appropriate packing parameters and the adding occurs above the melting temperature of the lipid. The method described by Keller et al, U.S. Pat. No. 6,610,322 is incorporated into this description. Most, if not all, known liposome suspensions are not thermodynamically stable. Instead, the liposomes in known suspensions are kinetically trapped into higher energy states by the energy used in their formation. Energy may be provided as heat, sonication, extrusion, or homogenization. Since every high-energy state tries to lower its free energy, known liposome formulations experience problems with aggregation, fusion, sedimentation and leakage of liposome associated material. A thermodynamically stable liposome formulation which could avoid some of these problems is therefore desirable.

The present embodiment prefers liposome suspensions which are thermodynamically stable at the temperature of formation. The formulation of such suspensions is achieved by employing a composition of lipids having several fundamental properties. First, the lipid composition must have packing parameters which allow the formation of liposomes. Second, as part of the head group, the lipid should include polyethyleneglycol (PEG) or any polymer of similar properties which sterically stabilizes the liposomes in suspension. Third, the lipid must have a melting temperature which allows it to be in liquid form when mixed with an aqueous solution.

By employing lipid compositions having the desired fundamental properties, little or no energy need be added when mixing the lipid and an aqueous solution to form liposomes. When mixed with water, the lipid molecules disperse and self assemble as the system settles into its natural low free energy state. Depending on the lipids used, the lowest free energy state may include small unilamellar vesicle (SUV) liposomes, multilamellar vesicle (MLV) liposomes, or a combination of SUVs and MLVs.

In one aspect, the invention includes a method of preparing liposomes. The method comprises providing an aqueous solution; providing a lipid solution, where the solution has a packing parameter measurement of Pₐ (Pₐ, references the surface packing parameter) between about 0.84 and 0.88, a Pᵥ (Pᵥ references the volume packing parameter) between about 0.88 and 0.93, (See, D. D. Lasic, Liposomes, From Physics to Applications, Elsevier, p. 51 1993), and where at least one lipid in the solution includes a polyethyleneglycol (PEG) chain; and combining the lipid solution and the aqueous solution. The PEG chain preferably has a molecular weight between about 300 Daltons and 5000 Daltons. Kinetic energy, such as shaking or vortexing, may be provided to the lipid solution and the aqueous solution. The lipid solution may comprise a single lipid. The lipid may comprise dioleolylglycerol-PEG-12, either alone or as one of the lipids in a mixture. The method may further comprise providing an active compound, in this case glutathione (reduced); and combining the active compound with the lipid solution and the aqueous solution.

The low molecular weight in the preferred embodiments more effectively deliver the liposomally encapsulated reduced glutathione in active reduced form as needed and thus result in the surprising effect of the invention. The absorption into cells is a particular advantage of the preferred embodiment of the invention.

### Further Examples 6

Formulation for Topical application of liposomally encapsulated reduced glutathione

A topical cream or lotion containing reduced glutathione in a self-forming liposome sold under the brand name “QuSome” ® by Biozone Laboratories, Inc. of Pittsburgh, Calif. is another preferred embodiment. The Qusome self-forming liposome can be formed containing reduced liposomally encapsulated glutathione in a concentration of 5% reduced glutathione encapsulated in the liposome. Most liposomes use energy provided as heat, sonication, extrusion, or homogenization for their formation, which gives them a high energy state. Some liposome formulations can experience problems with aggregation, fusion, sedimentation and leakage of liposome associated material which this invention seeks to minimize and does minimize. The Qusome is a more thermodynamically stable liposome formulation. The Qusome self-forming liposome is self-forming at room temperature which that the mixing of the lipid and an aqueous lipid containing solution avoids alteration of the contents by heating. The resulting liposome is in a low free energy state so it remains stable and reproducible. The formulation of this embodiment is reviewed in example 3. The methods of manufacture described in Keller et al U.S. Pat. No. 6,958,160 and U.S. Pat. No. 7,150,883 are incorporated in this description. The most important details of that manufacturing are as follows:

The lipids used to form the lipid vesicles and liposomes in the present formulations can be naturally occurring lipids, synthetically made lipids or lipids that are semisynthetic. Any of the art known lipid or lipid like substances can be used to generate the compositions of the present invention. These include, but are not limited to, lecithin, ceramides, phosphatidylethanolamine, phosphotidylcholine, phosphatidylserine, cardiolipin and the like. Such lipid components for the preparation of lipid vesicles are well known in the art, for example see U.S. Pat. No. 4,485,954, and “Liposome Technology”, 2nd Ed, Vol. I (1993) G. Gregoriadis ed., CRC Press, Boca Raton, Fla.

Lipids with these properties that are particularly preferred in the present formulations include phospholipids, particularly highly purified, unhydrogenated lecithin containing high concentrations of phosphotidylcholine, such as that available under the trade name Phospholipon 90 from American Lecithin, or Nattermann Phospholipid, 33 Turner Road, Danbury, Conn. 06813-1908.

In formulating the liposomes, in one aspect, the invention includes a method of preparing liposomes. The method comprises providing an aqueous solution; providing a lipid solution, where the solution has a Pₐ between about 0.84 and 0.88, a Pᵥ between about 0.88 and 0.93, and where at least one lipid in the solution includes a polyethyleneglycol (PEG) chain; and combining the lipid solution and the aqueous solution. The PEG chain preferably has a molecular weight between about 300 Daltons and 5000 Daltons. Kinetic energy, such as shaking or vortexing, may be provided to the lipid solution and the aqueous solution. The lipid solution may comprise a single lipid. The lipid may comprise dioleolyglycerol-PEG-12, either alone or as one of the lipids in a mixture. The method may further comprise providing an active compound; and combining the active compound with the lipid solution and the aqueous solution.

In another aspect, the invention includes a liposome suspension. The suspension comprises one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain. The PEG chain preferably has a molecular weight between about 300 Daltons and 5000 Daltons. The suspension may comprise a single lipid. The lipid may comprise dioleolylglycerol-PEG-12. The suspension may further comprise an active compound, which may be selected from the group described above.

In another aspect, the invention includes a composition for combining with an aqueous solution to form a liposome suspension. The composition comprises one or more lipids, where the lipids as an aggregate have a Pₐ between about 0.84 and 0.88, a Pᵥ, between about 0.88 and 0.93 and a melting temperature of between about 0 to 100 degrees centigrade; and where at least one lipid includes a polyethyleneglycol (PEG) chain. The PEG chain preferably has a molecular weight between about 300 Daltons and 5000 Daltons. The composition may comprise a single lipid. The composition may comprise dioleolylglycerol-PEG 12. The composition may further comprise an active compound selected from the group above. The composition may be provided in a sealed container, where the container also contains an inert gas to prevent oxidative degradation.

In another aspect, the invention includes a method of intravenously administering a therapeutic compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing an active compound; providing an aqueous solution; combining the composition, compound and solution to form a liposome suspension; and administering the liposome suspension intravenously. The method may further comprise providing kinetic energy to the liposome suspension. The method may also include providing the composition in a sealed container containing an inert gas. The PEG chain preferably has a molecular weight between about 300 Daltons and 5000 Daltons. The composition may comprise a single lipid. The lipid may comprise dioleolylglycerol-PEG-12. The active compound may be selected from the group above.

In another aspect, the invention includes a method of solubilizing an active compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing the active compound; providing an aqueous solution; and combining the active compound, the lipid and the aqueous solution to form a liposome suspension. The method may further comprise providing kinetic energy to the liposome suspension. The method may include providing the composition in a sealed container containing an inert gas. The PEG chain preferably has a molecular weight between about 300 Daltons and 5000 Daltons. The composition may comprise, a single lipid. The lipid may comprise dioleolylglycerol-PEG-12. The active compound may be selected from the group above.

In another aspect, the invention includes a method of orally administering a therapeutic compound. The method comprises providing a composition including one or more lipids, where the lipids as an aggregate have a Pₐ between about 0.84 and 0.88, a Pᵥ between about 0.88 and 0.93 and a melting temperature of between about 0 to 100 degrees centigrade; and where at least one lipid includes a polyethyleneglycol (PEG) chain; providing an active compound; providing an aqueous solution; combining the composition, compound and solution to form a liposome suspension; and administering the liposome suspension orally in the form selected from the group comprising a two piece hard gelatin capsule, a soft gelatin capsule, or drops.

The compositions may be administered topically, inter-orally, vaginally or rectally.

PEG-12 Glyceryl Dioleate was obtained from Global 7 (New Jersey) for the following formulations. This can be substituted for the lecithin w/w % as needed to accomplish the formulation, or applied as set forth below.

In the following formulations, the “set percentage” w/w % of reduced glutathione is selected from 3.3%, 4%, 5%, 6%, 7%, 7.5%, 8%, 8.5% or 9% or amounts approximately to those percentages.

### Example 3A

Spontaneous Liposomes for Intravenously Administering Therapeutic Compounds or for a Spray or Drink

A set percentage of reduced glutathione is dissolved in a sufficient amount of the solvent PEG-12 Glyceryl Dioleate, also called dioleolylglycerol-PEG 12, (either referred to as “PEGDO”) and gently mixed for about 5 minutes. A sufficient amount of PEGDO should be about 10% w/w. Deionized water is slowly added to the solution. Ingredients other than deionized water, the reduced glutathione and the PEGDO may be added such as preferably 0.1% w/w potassium sorbate and then the final amount of deionized water added is that amount which is necessary to have the percentages add up to 100% w/w. Taste or other flavor-masking ingredients could also be added before the deionized water is brought up to 100% w/w. Although taste ingredients can be added before or after the liposomal encapsulation formulation, the preferable mode is to add flavor or other taste masking ingredients after liposomal encapsulation formulation, and they may be ingredients such as corn syrup, honey, sorbitol, sugar, saccharin, stevia, aspartame, citrus seed extract, natural peppermint oil, menthol, synthetic strawberry flavor, orange flavor, chocolate, or vanilla flavoring in concentrations from about 0.01 to 10%. The inventor has preferably used citrus seed extract.

### Example 3B

Spontaneous Liposomes for Intravenously Administered Therapeutic Compound and as a Drug Solubilization Vehicle for use in Spray or Drink

A set percentage of reduced glutathione is mixed with a sufficient amount of PEG-12 Glyceryl Dioleate, also called dioleolylglycerol-PEG 12, (either referred to as “PEGDO”) to bring the reduced glutathione into solution by vortexing and sonication for 10 minutes. A sufficient amount of PEGDO should be about 5% w/w. Deionized water is added and gently mixed. Ingredients other than deionized water, the reduced glutathione and the PEGDO may be added such as preferably 0.1% w/w potassium sorbate and then the final amount of deionized water added is that amount which is necessary to have the percentages add up to 100% w/w. Ingredients other than deionized water, the reduced glutathione and the PEGDO may be added such as preferably 0.1% w/w potassium sorbate and then the final amount of deionized water added is that amount which is necessary to have the percentages add up to 100% w/w. Taste ingredients or other flavor masking ingredients could also be added before the deionized water is brought up to 100% w/w. Although taste ingredients can be added before or after the liposomal formulation, the preferable mode is to add flavor or other taste masking ingredients after liposomal formulation, and they may be ingredients such as corn syrup, honey, sorbitol, sugar, saccharin, stevia, aspartame, citrus seed extract, natural peppermint oil, menthol, synthetic strawberry flavor, orange flavor, chocolate, or vanilla flavoring in concentrations from about 0.01 to 10%. The inventor has preferably used citrus seed extract.

The QuSome self-forming liposome uses polyethyleneglycol (PEG) is a steric stabilizer and the resulting liposome is of a moderate size, 150 nm-250 nm. The combination of 150 nm-250 nm size and the PEG component is known to create long circulating liposomes. The size of the QuSome self-forming liposome allows them to be sterile filtered. These attributes allow a secondary advantage of the invention by the QuSome liposome encapsulating a radionuclide useful for targeting tumors with either diagnostic radionuclides or therapeutic radionuclides. The QuSome self-forming liposome is of such as size and the presence of the steric stability with PEG results in long circulation time and an increased accumulation in the fine trabecular mesh of blood vessels supplying growing tumors. This characteristic allows for improved diagnostics as more radionuclide accumulates around the tumor improving the image of scans. This characteristic of accumulating in the trabecular mesh of blood vessels leading to tumors also leads to an improved therapeutic. The accumulation of QuSome self-forming liposomes in the blood vessel supply to tumors increases the radiation dosing to this area, creating damage to the tumor blood vessels creating an anti-angiogenic effect, resulting in a decreased supply of blood to the tumor and leading to death of tumor cells.

The concentration of liposomally encapsulated glutathione in the liposomes resulting from the Qusome formulation is 5% for topical application. It is possible to use the Qusome technology in creating an oral formulation also and the 8.25% glutathione in w/w concentration encapsulated in the liposome may be used in the oral formulation.

Thus the invention in one aspect is a method for preventing or reversing the formation of autophagic stromal cells in a tissue that has become cancer-prone due to increased oxidation stress in the tissue or the tissue micro-environment by orally administering, to a patient having tissue that has become cancer-prone, a dose of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM, and where such administering raises the level of reduced glutathione within cancer stromal cells.

Another aspect of the invention is a method for preventing or reversing the formation of autophagic stromal cells in a tissue that has become cancer-prone due to increased oxidation stress in the tissue or the tissue micro-environment, by orally administering, to a patient having tissue that has become cancer-prone, liposome-encapsulated reduced glutathione at least daily, where such administration raises the level of reduced glutathione within cancer stromal cells by at least 30% percent.

Another aspect of the invention is a method for enhancing the macrophage cell function in a tissue that has become cancer-prone due to increased oxidation stress in the tissue or the tissue micro-environment by orally administering, to a patient having tissue that has become cancer-prone, a dose of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM, where such administering raises the level of reduced glutathione within said macrophage cells.

Another aspect of the invention is a method for enhancing the macrophage function in a tissue that has become cancer-prone due to increased oxidation stress in the tissue or the tissue micro-environment by orally administering, to a patient having tissue that has become cancer-prone, liposome-encapsulated reduced glutathione at least daily, where such administration raises the level of GSH within said macrophage cells by at least 30%.

In radiation, or in chemotherapy which functions as a radiation mimic in terms of causing oxidative stress and generating free radicals, there is evidence of systemic oxidation in the blood of cancer-afflicted patients reflecting there is damage to peritumor cells and tissue. The invention functions to bring the peritumor cells to normal redox balance by supplying the liposomally encapsulated reduced glutathione intracellularly.

Thus, one aspect of the invention is a method of reducing undesirable cellular damage from radiation or chemotherapy treatment to a cancer patient by, beginning prior to the treatment, orally administering liposome-encapsulated reduced glutathione either at least twice daily to the patient for a period of at least 3 days or at least once daily for seven days, where such administration raises the level of GSH within peritumor stromal cells of the patient by at least 30 percent. A further refinement of this method is where each dose of such daily administration is between 6 mg/kg and 36 mg/kg weight of a patient of liposome-encapsulated reduced glutathione. Yet another refinement is where the liposome-encapsulated glutathione is administered in the form of a gel cap.

Another aspect of the invention is the use of a composition to prevent or reverse the formation of autophagic stromal cells in a tissue that has become cancer-prone due to increased oxidation stress in the tissue or the tissue micro-environment treat, said composition having liposomally encapsulated reduced glutathione in the percentage of 8.25% w/w.

Another aspect of the invention is the use of a composition to prevent or reverse the formation of autophagic stromal cells in a tissue that has become cancer-prone due to increased oxidation stress in the tissue or the tissue micro-environment treat, said composition having liposomally encapsulated reduced glutathione in the percentage of 8.5% w/w.

Another aspect of the invention is a method for the prevention of the recurrence of cancer using oral liposomally encapsulated reduced glutathione to maintain the presence and normal function of caveolin in fibroblast and other cells, by orally administering, to a patient having tissue that has become cancer-prone, liposome-encapsulated reduced glutathione at least daily, where such administration raises the level of reduced glutathione to maintain the presence and normal function of caveolin in fibroblast cells to diminish their glycolytic support for epithelial cancer cells, thus preventing the conversion of a fibroblast to an autophagic tumor stromal cells.

Another aspect of the invention is a method for the restoration of altered tumor stromal cells and peri-tumor fibroblasts to more normal mitochondrial function for these cells, by administering, to a patient having tissue that has become cancer-prone, a dose of a reduced glutathione stabilized and encapsulated in a liposomal pharmaceutical carrier capable of being ingested orally at least daily, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM, where such administration raises the level of GSH.

Another aspect of the invention is a method for enhancing the macrophage cell function in a tissue that has become cancer-prone due to increased oxidation stress in the tissue or the tissue micro-environment, by administering, to a patient having tissue that has become cancer-prone, a gel capsule and encapsulating with said gel capsule glutathione (reduced) said glutathione (reduced) being stabilized and encapsulated in a liposomal pharmaceutical carrier capable of being ingested orally, and capable of delivering glutathione (reduced) in a physiologically active state to improve symptoms in disease states by transfer of the glutathione (reduced) into animal cells, where the concentration of reduced glutathione in the entrapped aqueous space of the liposomes is at least about 123 mM, and where such administering raises the level of reduced glutathione within said macrophage cells. A further aspect is to use lecithin encapsulated within the gel capsule. A further aspect is to use up to 15-20% water encapsulated within the gel capsule. A further aspect is to use glycerin encapsulated within the gel capsule. A further aspect is to use sorbitan oleate encapsulated within the gel capsule. A further aspect is to use polysorbate 20 encapsulated within the gel capsule. A further aspect is to use potassium sorbate encapsulated within the gel capsule.

Another aspect is to use a gel capsule to prevent or reverse the formation of autophagic stromal cells in a tissue that has become cancer-prone due to increased oxidation stress in the tissue or the tissue micro-environment treat, by delivering, to a patient having tissue that has become cancer-prone, glutathione orally via a gel capsule including reduced glutathione encapsulated in a liposomal pharmaceutical carrier within said gel capsule where the concentration of reduced glutathione in the entrapped aqueous space of the liposomes is at least about 123 mM. The further aspects of the prior paragraph can then be utilized in conjunction with this use.

Another aspect is the use of a composition to prevent or reverse the formation of autophagic stromal cells in a tissue that has become cancer-prone due to increased oxidation stress in the tissue or the tissue micro-environment treat, said composition having liposomally encapsulated reduced glutathione in the percentage of 8.25% w/w.

Another aspect is the use of a composition to prevent or reverse the formation of autophagic stromal cells in a tissue that has become cancer-prone due to increased oxidation stress in the tissue or the tissue micro-environment treat, said composition having liposomally encapsulated reduced glutathione in the percentage of 8.5% w/w.

Another aspect is a method for the prevention of the recurrence of cancer using oral liposomally encapsulated reduced glutathione to maintain the presence and normal function of caveolin in fibroblast and other cells, by orally administering, to a patient having tissue that has become cancer-prone, liposome-encapsulated reduced glutathione at least daily, where such administration raises the level of reduced glutathione to maintain the presence and normal function of caveolin in fibroblast cells to diminish their glycolytic support for epithelial cancer cells, thus preventing the conversion of a fibroblast to an autophagic tumor stromal cells.

Another aspect is a method for the restoration of altered tumor stromal cells and peri-tumor fibroblasts to more normal mitochondrial function for these cells, by administering, to a patient having tissue that has become cancer-prone, a dose of a reduced glutathione stabilized and encapsulated in a liposomal pharmaceutical carrier capable of being ingested orally at least daily, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM, where such administration raises the level of GSH.

Another aspect of the invention is a pharmaceutical composition for preventing or reversing the effects of cancer-prone tissue comprising a therapeutic dose of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM, and the composition has sodium bicarbonate in a range of 1.5% w/w to 8.5% w/w, and the percentage of water/weight of the sum of the percentage of liposomally encapsulated reduced glutathione plus the percentage of water/weight of sodium bicarbonate in a dose is 1.5% w/w to 8.5% w/w.

Another aspect of the invention is a pharmaceutical composition for preventing or reversing the effects of cancer-prone tissue comprising a therapeutic dose of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM, and the percentage of water/weight sodium bicarbonate in a dose is from 1.5% w/w to 8.5% w/w.

Another aspect of the invention is a method of the prevention of weight loss and wasting associated with cancer progression and metastasis, by orally administering, to a patient having tissue that has become cancer-prone, a dose of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM.

Another aspect of the invention is a pharmaceutical composition for preventing or reversing the effects of cancer-prone tissue comprising a therapeutic dose of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM, and a daily dose of dichloroacetic acid (DCA) in a range from 10 mg/kg to 100 mg/kg. A further aspect of this composition is to include triiodothyronine (cytomel) in a range of 5 micrograms to 15 micrograms. A further aspect of this composition is to include caffeine.

Another aspect of the invention is an anti-cancer pharmaceutical composition of EDTA and a liposomally encapsulated formulation of reduced glutathione, comprising a therapeutic dose of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM, and EDTA in a range of 100 mg to 3 grams in a single dose administered every other day.

Another aspect of the invention is a method for preventing or reversing the formation of autophagic stromal cells in a tissue that has become cancer-prone due to increased oxidation stress in the tissue or the tissue micro-environment, by administering, to a patient having tissue that has become cancer-prone, two therapeutic doses per day of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM, and administering arginine in two doses per day.

Another aspect of the invention is a method for preventing or reversing the formation of autophagic stromal cells in a tissue that has become cancer-prone due to increased oxidation stress in the tissue or the tissue micro-environment, by administering, to a patient having tissue that has become cancer-prone, two therapeutic doses per day of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM and by administering arginine in two doses per day.

Another aspect of the invention is a method for preventing or reversing the formation of autophagic stromal cells in a tissue that has become cancer-prone due to increased oxidation stress in the tissue or the tissue micro-environment, by administering, to a patient having tissue that has become cancer-prone, two therapeutic doses in a range of ½ teaspoon to 4 teaspoons per dose of GSNO stabilized and encapsulated in a liposomal pharmaceutical carrier capable of being ingested orally, and capable of delivering GSNO in a physiologically active state into cells.

Another aspect of the invention is a method for preventing or reversing the formation of autophagic stromal cells in a tissue that has become cancer-prone due to increased oxidation stress in the tissue or the tissue micro-environment, by administering, to a patient having tissue that has become cancer-prone, two therapeutic doses per day of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM, and administering, to a patient having tissue that has become cancer-prone, two therapeutic doses per day in a range of ½ teaspoon to 4 teaspoons per dose of GSNO stabilized and encapsulated in a liposomal pharmaceutical carrier capable of being ingested orally, and capable of delivering GSNO in a physiologically active state into cells.

Another aspect of the invention is an anti-cancer composition comprising D-Alpha-tocopherol succinate encapsulated in liposomes ranging in size from 20 nm to 10 microns at a concentration of 400 mg of D-Alpha-tocopherol succinate per 5 cc of liposomal liquid and a therapeutic dose of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM.

Another aspect of the invention is a method of reducing the cancer promoting effects of mycotoxins, by administering an antifungal agent, and by administering, to a patient having tissue that has become cancer-prone, a therapeutic dose of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM. A further aspect of t his method is using voriconazole as the anti-fungal agent.

Another aspect of the invention is the use of a medicament comprising an antifungal agent, and a therapeutic dose of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM.

### REFERENCES

1. SEER. Seer Stat Fact Sheets. Surveillance Epidemiology and End Results [serial on the Internet]. 2010; 2010: Available from: http://seer.cancer.gov/statfacts/html/all.html#prevalence.
2. Warburg O, Negelein E. The Metabolism of Tumors in the Body. Journal of General Physiology. 1927; 8(6):519-30.
3. Levine A J, Puzio-Kuter A M. The control of the metabolic switch in cancers by oncogenes and tumor suppressor genes. Science (New York, N.Y. 2010; 330(6009):1340-4. Cited in PubMed; 21127244.
4. Seyfried T N, Shelton L M. Cancer as a metabolic disease. Nutrition & metabolism. 2010; 7: 7. Cited in PubMed; 20181022 http://www.ncbi.nlm.nih.gov/pmc/articles/PMC2845135/?tool=pubmed.
5. Roskelley R C, Mayer N, Horwitt B N, Salter W T. Studies in Cancer. Vii. Enzyme Deficiency in Human and Experimental Cancer. The Journal of clinical investigation. 1943; 22(5):743-51. Cited in PubMed; 16695058 http://www.ncbi.nlm.nih.gov/pmc/articles/PMC435291/?tool=pubmed.
6. Szent-Gyorgyi A. The living state and cancer. Proceedings of the National Academy of Sciences of the United States of America. 1977; 74(7):2844-7. Cited in PubMed; 268635 http://www.ncbi.nlm.nih.gov/pmc/articles/PMC431314/pdf/pnas00029-0256.pdf.
7. Kolata G. HOPE IN THE LAB: A special report.; A Cautious Awe Greets Drugs That Eradicate Tumors in Mice. New York Times [serial on the Internet]. 1998; (May 3): Available from: http://www.nytimes.com/1998/05/03/us/hope-lab-special-report-cautious-awe-greets-drugs-that-eradicate-tumors-mice.html.
8. Lisanti M P, Martinez-Outschoorn U E, Chiavarina B, Pavlides S, Whitaker-Menezes D, Tsirigos A, et al. Understanding the “lethal” drivers of tumor-stroma co-evolution: Emerging role(s) for hypoxia, oxidative stress and autophagy/mitophagy in the tumor micro-environment. Cancer biology & therapy. 2010; 10(6). Cited in PubMed; 20861671 http://www.landesbioscience.com/journals/cbt/article/13370/.
9. Liao D, Luo Y, Markowitz D, Xiang R, Reisfeld R A. Cancer associated fibroblasts promote tumor growth and metastasis by modulating the tumor immune microenvironment in a 4T1 murine breast cancer model. PLoS One. 2009; 4(11):e7965. Cited in PubMed; 19956757 http://www.plosone.org/article/info:doi/10.1371/journal.pone.0007965.
10. Delimaris I, Faviou E, Antonakos G, Stathopoulou E, Zachari A, Dionyssiou-Asteriou A. Oxidized LDL, serum oxidizability and serum lipid levels in patients with breast or ovarian cancer. Clinical biochemistry. 2007; 40(15):1129-34. Cited in PubMed; 17673194.
11. Welters M J, Fichtinger-Schepman A M, Baan R A, Flens M J, Scheper R J, Braakhuis B J. Role of glutathione, glutathione S-transferases and multidrug resistance-related proteins in cisplatin sensitivity of head and neck cancer cell lines. British journal of cancer. 1998; 77(4):556-61. Cited in PubMed; 9484811 http://www.ncbi.nlm.nih.gov/pmc/articles/PMC2149938/pdf/brjcancer00080-0046.pdf.
12. Wikipedia. Warburg effect. Wikipedia [serial on the Internet]. 2010: Available from: http://en.wikipedia.org/wiki/Warburg_effect.
13. Papandreou I, Goliasova T, Denko N C. Anti-cancer drugs that target metabolism, is dichloroacetate the new paradigm? Int J. Cancer. 2010. Cited in PubMed; 20957634.
14. Michelakis E D, Webster L, Mackey J R. Dichloroacetate (DCA) as a potential metabolic-targeting therapy for cancer. British journal of cancer. 2008; 99(7):989-94. Cited in PubMed; 18766181 http://www.ncbi.nlm.nih.gov/pmc/articles/PMC2567082/?tool=pubmed.
15. Pavlides S, Tsirigos A, Migneco G, Whitaker-Menezes D, Chiavarina B, Flomenberg N, et al. The autophagic tumor stroma model of cancer: Role of oxidative stress and ketone production in fueling tumor cell metabolism. Cell cycle (Georgetown, Tex. 2010; 9(17):3485-505. Cited in PubMed; 20861672 http://www.landesbioscience.com/journals/cc/article/PavlidesCC9-17-2.pdf.
16. Tang R, Beuvon F, Ojeda M, Mosseri V, Pouillart P, Scholl S. M-CSF (monocyte colony stimulating factor) and M-CSF receptor expression by breast tumour cells: M-CSF mediated recruitment of tumour infiltrating monocytes? Journal of cellular biochemistry. 1992; 50(4):350-6. Cited in PubMed; 1334964.
17. Dunn G P, Old L J, Schreiber R D. The immunobiology of cancer immunosurveillance and immunoediting. Immunity. 2004; 21(2):137-48. Cited in PubMed; 15308095.
18. Lamagna C, Aurrand-Lions M, Imhof B A. Dual role of macrophages in tumor growth and angiogenesis. Journal of leukocyte biology. 2006; 80(4):705-13. Cited in PubMed; 16864600 http://www.jleukbio.org/content/80/4/705.1ong.
19. Maekawa H, Iwabuchi K, Nagaoka I, Watanabe H, Kamano T, Tsurumaru M. Activated peritoneal macrophages inhibit the proliferation of rat ascites hepatoma AH-130 cells via the production of tumor necrosis factor-alpha and nitric oxide. Inflamm Res. 2000; 49(10):541-7. Cited in PubMed; 11089907.
20. Boger R H, Ron E S. L-Arginine improves vascular function by overcoming deleterious effects of ADMA, a novel cardiovascular risk factor. Ahern Med Rev. 2005; 10(1):14-23. Cited in PubMed; 15771559.
21. Vallance P, Leone A, Calver A, Collier J, Moncada S. Endogenous dimethylarginine as an inhibitor of nitric oxide synthesis. J Cardiovasc Pharmacol. 1992; 20 Suppl 12:S60-2. Cited in PubMed; 1282988.
22. Cooke J P. Asymmetrical dimethylarginine: the Uber marker? Circulation. 2004; 109(15):1813-8. Cited in PubMed; 15096461.
23. Abuja P M, Albertini R. Methods for monitoring oxidative stress, lipid peroxidation and oxidation resistance of lipoproteins. Clin Chim Acta. 2001; 306(1-2):1-17. Cited in PubMed; 11282089.
24. Beckman J S, Koppenol W H. Nitric oxide, superoxide, and peroxynitrite: the good, the bad, and ugly. Am J. Physiol. 1996; 271(5 Pt 1):C1424-37. Cited in PubMed; 8944624.
25. Halliwell B. What nitrates tyrosine? Is nitrotyrosine specific as a biomarker of peroxynitrite formation in vivo? FEBS letters. 1997; 411(2-3):157-60. Cited in PubMed; 9271196.
26. Pryor W A, Squadrito G L. The chemistry of peroxynitrite: a product from the reaction of nitric oxide with superoxide. Am J. Physiol. 1995; 268(5 Pt 1):L699-722. Cited in PubMed; 7762673.
27. Taddei S, Virdis A, Ghiadoni L, Salvetti G, Bernini G, Magagna A, et al. Age-related reduction of NO availability and oxidative stress in humans. Hypertension. 2001; 38(2):274-9. Cited in PubMed; 11509489.
28. Miyazaki H, Matsuoka H, Cooke J P, Usui M, Ueda S, Okuda S, et al. Endogenous nitric oxide synthase inhibitor: a novel marker of atherosclerosis. Circulation. 1999; 99(9):1141-6. Cited in PubMed; 10069780.
29. Perticone F, Sciacqua A, Maio R, Perticone M, Maas R, Boger R H, et al. Asymmetric dimethylarginine, L-arginine, and endothelial dysfunction in essential hypertension. J Am Coll Cardiol. 2005; 46(3):518-23. Cited in PubMed; 16053968.
30. Boger R H, Sydow K, Borlak J, Thum T, Lenzen H, Schubert B, et al. LDL cholesterol upregulates synthesis of asymmetrical dimethylarginine in human endothelial cells: involvement of S-adenosylmethionine-dependent methyltransferases. Circulation research. 2000; 87(2):99-105. Cited in PubMed; 10903992.
31. Ito A, Tsao P S, Adimoolam S, Kimoto M, Ogawa T, Cooke J P. Novel mechanism for endothelial dysfunction: dysregulation of dimethylarginine dimethylaminohydrolase. Circulation. 1999; 99(24):3092-5. Cited in PubMed; 10377069.
32. Mills C D, Shearer J, Evans R, Caldwell M D. Macrophage arginine metabolism and the inhibition or stimulation of cancer. J. Immunol. 1992; 149(8):2709-14. Cited in PubMed; 1401910.
33. Mills C D. Macrophage arginine metabolism to ornithine/urea or nitric oxide/citrulline: a life or death issue. Critical reviews in immunology. 2001; 21(5):399-425. Cited in PubMed; 11942557.
34. Martinez-Outschoorn U E, Trimmer C, Lin Z, Whitaker-Menezes D, Chiavarina B, Zhou J, et al. Autophagy in cancer associated fibroblasts promotes tumor cell survival: Role of hypoxia, HIFI induction and NFkappaB activation in the tumor stromal microenvironment. Cell cycle (Georgetown, Tex. 2010; 9(17):3515-33. Cited in PubMed; 20855962 http://www.landesbioscience.com/journals/cc/article/OutschoomCC9-17.pdf.
35. Nathan C F, Arrick B A, Murray H W, DeSantis N M, Cohn Z A. Tumor cell anti-oxidant defenses. Inhibition of the glutathione redox cycle enhances macrophage-mediated cytolysis. The Journal of experimental medicine. 1981; 153(4):766-82. Cited in PubMed; 7252413 http://www.ncbi.nlm.nih.gov/pmc/articles/PMC2186135/pdf/766.pdf.
36. Ortega A, Carretero J, Obrador E, Estrela J M. Tumoricidal activity of endothelium-derived NO and the survival of metastatic cells with high GSH and Bcl-2 levels. Nitric Oxide. 2008; 19(2):107-14. Cited in PubMed; 18474263.
37. Zeevalk G D, Bernard L P, Guilford F T. Liposomal-glutathione provides maintenance of intracellular glutathione and neuroprotection in mesencephalic neuronal cells. Neurochemical research. 2010; 35(10):1575-87. Cited in PubMed; 20535554.
38. Lyons J, Rauh-Pfeiffer A, Ming-Yu Y, Lu X M, Zurakowski D, Curley M, et al. Cysteine metabolism and whole blood glutathione synthesis in septic pediatric patients. Critical care medicine. 2001; 29(4):870-7. Cited in PubMed; 11373484.
39. Darmaun D, Smith S D, Sweeten S, Hartman B K, Welch S, Mauras N. Poorly controlled type 1 diabetes is associated with altered glutathione homeostasis in adolescents: apparent resistance to N-acetylcysteine supplementation. Pediatric diabetes. 2008; 9(6):577-82. Cited in PubMed; 19067892.
40. Ionescu J G, Novotny J, Stejskal V, Latsch A, Blaurock-Busch E, Eisenmann-Klein M. Increased levels of transition metals in breast cancer tissue. Neuro endocrinology letters. 2006; 27 Suppl 1:36-9. Cited in PubMed; 16804515.
41. Papadopoulou E, Tripsianis G, Anagnostopoulos K, Tentes I, Kakolyris S, Galazios G, et al. Significance of serum tumor necrosis factor-alpha and its combination with HER-2 codon 655 polymorphism in the diagnosis and prognosis of breast cancer. The International journal of biological markers. 2010; 25(3):126-35. Cited in PubMed; 20872355.
42. Wang H, Wang A X, Barrett E J. Caveolin-1 is Required for Vascular Endothelial Insulin Uptake. Am J Physiol Endocrinol Metab. 2010. Cited in PubMed; 20959538.
43. Ishii Y, Partridge C A, Del Vecchio P J, Malik A B. Tumor necrosis factor-alpha-mediated decrease in glutathione increases the sensitivity of pulmonary vascular endothelial cells to H202. The Journal of clinical investigation. 1992; 89(3):794-802. Cited in PubMed; 1541673.
44. Kemik O, Sumer A, Kemik A S, Hasirci I, Purisa S, Dulger A C, et al. The relationship among acute-phase response proteins, cytokines and hormones in cachectic patients with colon cancer. World journal of surgical oncology. 2010; 8:85. Cited in PubMed; 20920199 http://www.ncbi.nlm.nih.gov/pubmed/20920199.
45. Tijerina A J. The biochemical basis of metabolism in cancer cachexia. Dimens Crit Care Nurs. 2004; 23(6):237-43. Cited in PubMed; 15586034 http://journals.1ww.com/dccnjournal/fulltext/2004/11000/the_biochemical_basis_of_metabolism_in_cancer.1.aspx.
46. De Blaauw I, Deutz N E, Von Meyenfeldt M F. Metabolic changes in cancer cachexia—first of two parts. Clinical nutrition (Edinburgh, Scotland). 1997; 16(4):169-76. Cited in PubMed; 16844595.
47. Keller U. Pathophysiology of cancer cachexia. Support Care Cancer. 1993; 1(6):290-4. Cited in PubMed; 8156245.
48. Martinez-Outschoorn U E, Balliet R M, Rivadeneira D B, Chiavarina B, Pavlides S, Wang C, et al. Oxidative stress in cancer associated fibroblasts drives tumor-stroma co-evolution: A new paradigm for understanding tumor metabolism, the field effect and genomic instability in cancer cells. Cell cycle (Georgetown, Tex. 2010; 9(16):3256-76. Cited in PubMed; 20814239 http://www.landesbioscience.com/journals/cc/article/12553/.
49. Wang L, Lim E J, Toborek M, Hennig B. The role of fatty acids and caveolin-1 in tumor necrosis factor alpha-induced endothelial cell activation. Metabolism: clinical and experimental. 2008; 57(10):1328-39. Cited in PubMed; 18803934.
50. Liu P, Rudick M, Anderson R G. Multiple functions of caveolin-1. The Journal of biological chemistry. 2002; 277(44):41295-8. Cited in PubMed; 12189159 http://www.jbc.org/content/277/44/41295.full.
51. Thompson T C, Tahir S A, Li L, Watanabe M, Naruishi K, Yang G, et al. The role of caveolin-1 in prostate cancer: clinical implications. Prostate cancer and prostatic diseases. 2010; 13(1):6-11. Cited in PubMed; 19581923 http://www.medscape.com/viewarticle/718699.
52. Yang G, Addai J, Ittmann M, Wheeler T M, Thompson T C. Elevated Caveolin-1 Levels in African-American versus White-American Prostate Cancer. Clinical Cancer Research. 2000; 6(9):3430-3.
53. Ho C C, Huang P H, Huang H Y, Chen Y H, Yang P C, Hsu S M. Up-regulated caveolin-1 accentuates the metastasis capability of lung adenocarcinoma by inducing filopodia formation. The American journal of pathology. 2002; 161(5):1647-56. Cited in PubMed; 12414512 http://www.ncbi.nlm.nih.gov/pmc/articles/PMC1850800/?tool=pubmed.
54. Joo H J, Oh D K, Kim Y S, Lee K B, Kim S J. Increased expression of caveolin-1 and microvessel density correlates with metastasis and poor prognosis in clear cell renal cell carcinoma. BJU international. 2004; 93(3):291-6. Cited in PubMed; 14764125.
55. Kato K, Hida Y, Miyamoto M, Hashida H, Shinohara T, Itoh T, et al. Overexpression of caveolin-1 in esophageal squamous cell carcinoma correlates with lymph node metastasis and pathologic stage. Cancer. 2002; 94(4):929-33. Cited in PubMed; 11920460.
56. Tahir S A, Ren C, Timme T L, Gdor Y, Hoogeveen R, Morrisett J D, et al. Development of an immunoassay for serum caveolin-1: a novel biomarker for prostate cancer. Clin Cancer Res. 2003; 9(10 Pt 1):3653-9. Cited in PubMed; 14506154.
57. Tahir S A, Yang G, Ebara S, Timme T L, Satoh T, Li L, et al. Secreted caveolin-1 stimulates cell survival/clonal growth and contributes to metastasis in androgen-insensitive prostate cancer. Cancer Res. 2001; 61(10):3882-5. Cited in PubMed; 11358800 http://cancerres.aacrjournals.org/content/61/10/3882.1ong.
58. Ortega A D, Sanchez-Arago M, Giner-Sanchez D, Sanchez-Cenizo L, Willers I, Cuezva J M. Glucose avidity of carcinomas. Cancer letters. 2009; 276(2):125-35. Cited in PubMed; 18790562.
59. Fojo T, Bates S. Strategies for reversing drug resistance. Oncogene. 2003; 22(47):7512-23. Cited in PubMed; 14576855.
60. Tahir S A, Frolov A, Hayes T G, Mims M P, Miles B J, Lerner S P, et al. Preoperative serum caveolin-1 as a prognostic marker for recurrence in a radical prostatectomy cohort. Clin Cancer Res. 2006; 12(16):4872-5. Cited in PubMed; 16914574 http://clincancerres.aacrjournals.org/content/12/16/4872.1ong.
61. Gatenby R A, Gillies R J. Why do cancers have high aerobic glycolysis? Nat Rev Cancer. 2004; 4(11):891-9. Cited in PubMed; 15516961.
62. Shupe T, Sell S. Low hepatic glutathione S-transferase and increased hepatic DNA adduction contribute to increased tumorigenicity of aflatoxin B1 in newborn and partially hepatectomized mice. Toxicology letters. 2004; 148(1-2):1-9. Cited in PubMed; 15019083.
63. Munzarova M, Kovarik J. Is cancer a macrophage-mediated autoaggressive disease? Lancet. 1987; 1(8539):952-4. Cited in PubMed; 2882343.
64. Lu X, Kang Y. Cell fusion as a hidden force in tumor progression. Cancer Res. 2009; 69(22):8536-9. Cited in PubMed; 19887616 http://www.ncbi.nlm.nih.gov/pmc/articles/PMC2783941/?tool=pubmed.
65. Huysentruyt L C, McGrath M S. The role of macrophages in the development and progression of AIDS-related non-Hodgkin lymphoma. Journal of leukocyte biology. 2010; 87(4):627-32. Cited in PubMed; 20042471 http://www.ncbi.nlm.nih.gov/pmc/articles/PMC2858304/?tool=pubmed.
66. Morantz R A, Wood G W, Foster M, Clark M, Gollahon K. Macrophages in experimental and human brain tumors. Part 2: studies of the macrophage content of human brain tumors. Journal of neurosurgery. 1979; 50(3):305-11. Cited in PubMed; 422981.
67. Hooper D G. The Hidden Truth Of Mycotoxins. 37th Annual meeting Cancer Control Society [serial on the Internet]. 2009: Available from: http://www.cancercontrolsociety.com/forms/37thannual-program.pdf.
68. Cavin C, Delatour T, Marin-Kuan M, Holzhauser D, Higgins L, Bezencon C, et al. Reduction in antioxidant defenses may contribute to ochratoxin A toxicity and carcinogenicity. Toxicol Sci. 2007; 96(1):30-9. Cited in PubMed; 17110534.
69. Marin-Kuan M, Nestler S, Verguet C, Bezencon C, Piguet D, Mansourian R, et al. A Toxicogenomics Approach to Identify New Plausible Epigenetic Mechanisms of Ochratoxin A Carcinogenicity in Rat. Toxicol Sci. 2006; 89(1):120-34.
70. Cusumano V, Costa G B, Seminara S. Effect of aflatoxins on rat peritoneal macrophages. Applied and environmental microbiology. 1990; 56(11):3482-4. Cited in PubMed; 2176448 http://www.ncbi.nlm.nih.gov/pmc/articles/PMC184993/?tool=pubmed.
71. Iverson F, Campbell J, Clayson D, Hierlihy S, Labossiere E, Hayward S. Effects of antioxidants on aflatoxin-induced hepatic tumors in rats. Cancer letters. 1987; 34(2):139-44. Cited in PubMed; 3102038.
72. Wagner G, Frenzel H, Wefers H, Sies H. Lack of effect of long-term glutathione administration on aflatoxin B1-induced hepatoma in male rats. Chemico-biological interactions. 1985; 53(1-2):57-68. Cited in PubMed; 3922636.
73. Fukal L, Reisnerova H. Monitoring of aflatoxins and ochratoxin A in Czechoslovak human sera by immunoassay. Bulletin of environmental contamination and toxicology. 1990; 44(3):345-9. Cited in PubMed; 2328339.
74. Wangikar P B, Dwivedi P, Sinha N. Effect in rats of simultaneous prenatal exposure to ochratoxin A and aflatoxin B1. I. Maternal toxicity and fetal malformations. Birth defects research. 2004; 71(6):343-51. Cited in PubMed; 15617020.
75. Huysentruyt L C, Seyfried T N. Perspectives on the mesenchymal origin of metastatic cancer. Cancer metastasis reviews. 2010; 29(4):695-707. Cited in PubMed; 20839033 http://www.ncbi.nlm.nih.gov/pmc/articles/PMC2962789/?tool=pubmed.
76. Kuijpers S A, Coimbra M J, Storm G, Schiffelers R M. Liposomes targeting tumour stromal cells. Molecular membrane biology. 2010; 27(7):328-40. Cited in PubMed; 20939769.
77. Roehr B. Statins Show Antimicrobial Activity in Vitro. Medscape Today [serial on the Internet]. 2007; (September 30): Available from: http://www.medscape.com/viewarticle/563088.
78. Witschi A, Reddy S, Stofer B, Lauterburg B H. The systemic availability of oral glutathione. European journal of clinical pharmacology. 1992; 43(6):667-9. Cited in PubMed; 1362956.
79. Ozols R F. Pharmacologic reversal of drug resistance in ovarian cancer. Seminars in oncology. 1985; 12(3 Suppl 4):7-11. Cited in PubMed; 4048979.
80. Chen H H, Kuo M T. Role of glutathione in the regulation of Cisplatin resistance in cancer chemotherapy. Metal-based drugs. 2010; 2010. Cited in PubMed; 20885916 http://www.ncbi.nlm.nih.gov/pmc/articles/PMC2946579/?tool=pubmed.
81. Zhou S F. Structure, function and regulation of P-glycoprotein and its clinical relevance in drug disposition. Xenobiotica; the fate of foreign compounds in biological systems. 2008; 38(7-8):802-32. Cited in PubMed; 18668431.
82. Buda G, Ricci D, Huang C C, Favis R, Cohen N, Zhuang S H, et al. Polymorphisms in the multiple drug resistance protein 1 and in P-glycoprotein 1 are associated with time to event outcomes in patients with advanced multiple myeloma treated with bortezomib and pegylated liposomal doxorubicin. Annals of hematology. 2010; 89(11):1133-40. Cited in PubMed; 20532504 http://www.ncbi.nlm.nih.gov/pmc/articles/PMC2940014/?tool=pubmed.
83. ACS. Insulin Potentiated Therapy. Cancer Treatment Information [serial on the Internet]. 2010; American Cancer Society: Available from: http://www.cancer.org/Treatment/TreatmentsandSideEffects/ComplementaryandAlternativeMedi cine/PharmacologicalandBiologicalTreatment/insulin-potentiation-therapy.
84. Bump E A, Yu N Y, Brown J M. Radiosensitization of hypoxic tumor cells by depletion of intracellular glutathione. Science (New York, N.Y. 1982; 217(4559):544-5. Cited in PubMed; 7089580.
85. Hiraiwa K, Oka T, Yagi K. Effect of bleomycin on lipid peroxides, glutathione peroxidase and collagenase in cultured lung fibroblasts. J. Biochem. 1983; 93(4):1203-10. Cited in PubMed; 6190799.
86. Chattopadhyay A, Choudhury S, Chatterjee A. Modulation of the clastogenic activity of bleomycin by reduced-glutathione, glutathione-ester and buthionine sulphoximine. Mutagenesis. 1997; 12(4):221-5. Cited in PubMed; 9237765 http://mutage.oxfordjournals.org/cgi/reprint/12/4/221.pdf.
87. Chatterjee A, Jacob-Raman M, Mohapatra B. Potentiation of bleomycin-induced chromosome aberrations by the radioprotector reduced glutathione. Mutation research. 1989; 214(2):207-13. Cited in PubMed; 2477697.
88. Schwartz D R, Homanics G E, Hoyt D G, Klein E, Abernethy J, Lazo J S. The neutral cysteine protease bleomycin hydrolase is essential for epidermal integrity and bleomycin resistance. Proceedings of the National Academy of Sciences of the United States of America. 1999; 96(8):4680-5. Cited in PubMed; 10200322 http://www.ncbi.nlm.nih.gov/pmc/articles/PMC16392/?tool=pubmed.
89. Tsuruo T, Hamilton T C, Louie K G, Behrens B C, Young R C, Ozols R F. Collateral susceptibility of adriamycin-, melphalan- and cisplatin-resistant human ovarian tumor cells to bleomycin. Jpn J Cancer Res. 1986; 77(9):941-5. Cited in PubMed; 2429947.
90. Pani G, Galeotti T, Chiarugi P. Metastasis: cancer cell's escape from oxidative stress. Cancer metastasis reviews. 2010; 29(2):351-78. Cited in PubMed; 20386957

I claim:

1. A method for preventing or reversing the formation of autophagic stromal cells in a tissue that has become cancer-prone due to increased oxidation stress in the tissue or the tissue micro-environment, the method comprising:
orally administering, to a patient having tissue that has become cancer-prone, a dose of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM,
where such administering raises the level of reduced glutathione within cancer stromal cells.

2. The method according to claim 1, further comprising the following steps:
administering, to a patient having tissue that has become cancer-prone, two therapeutic doses per day of said reduced glutathione stabilized and encapsulated in a liposomal pharmaceutical carrier and;
administering arginine in two doses per day.

3. A method for preventing or reversing the formation of autophagic stromal cells in a tissue that has become cancer-prone due to increased oxidation stress in the tissue or the tissue micro-environment, the method comprising:
orally administering, to a patient having tissue that has become cancer-prone, liposome-encapsulated reduced glutathione at least daily, where such administration raises the level of reduced glutathione within cancer stromal cells by at least 30% percent.

4. A method for enhancing the macrophage cell function in a tissue that has become cancer-prone due to increased oxidation stress in the tissue or the tissue micro-environment, the method comprising:
orally administering, to a patient having tissue that has become cancer-prone, a dose of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM,
where such administering raises the level of reduced glutathione within said macrophage cells.

5. A method for enhancing the macrophage function in a tissue that has become cancer-prone due to increased oxidation stress in the tissue or the tissue micro-environment, the method comprising:
orally administering, to a patient having tissue that has become cancer-prone, liposome-encapsulated reduced glutathione at least daily, where such administration raises the level of reduced glutathione within said macrophage cells by at least 30%.

6. A method for enhancing the macrophage cell function in a tissue that has become cancer-prone due to increased oxidation stress in the tissue or the tissue micro-environment, the method comprising:
administering, to a patient having tissue that has become cancer-prone, a gel capsule; and
encapsulating with said gel capsule glutathione (reduced) said glutathione (reduced) being stabilized and encapsulated in a liposomal pharmaceutical carrier capable of being ingested orally, and capable of delivering glutathione (reduced) in a physiologically active state to improve symptoms in disease states by transfer of the glutathione (reduced) into animal cells,
where the concentration of reduced glutathione in the entrapped aqueous space of the liposomes is at least about 123 mM; and
where such administering raises the level of reduced glutathione within said macrophage cells

7. The method of claim 6, further comprising:
lecithin encapsulated within the gel capsule.

8. The method of claim 6, further comprising:
up to 15-20% water encapsulated within the gel capsule.

9. The method of claim 6, further comprising:
glycerin encapsulated within the gel capsule.

10. The method of claim 6, further comprising:
sorbitan oleate encapsulated within the gel capsule.

11. The method of claim 6, further comprising:
polysorbate 20 encapsulated within the gel capsule.

12. The method of claim 6, further comprising:
potassium sorbate encapsulated within the gel capsule.

13. A method for the prevention of the recurrence of cancer using oral liposomal reduced glutathione to maintain the presence and normal function of caveolin in fibroblast and other cells, the method comprising:
orally administering, to a patient having tissue that has become cancer-prone, liposome-encapsulated reduced glutathione at least daily, where such administration raises the level of reduced glutathione to maintain the presence and normal function of caveolin in fibroblast cells to diminish their glycolytic support for epithelial cancer cells, thus preventing the conversion of a fibroblast to an autophagic tumor stromal cell.

14. A method of prevention of weight loss and wasting associated with cancer progression and metastasis, comprising the following step:
orally administering, to a patient having tissue that has become cancer-prone, a dose of a reduced glutathione stabilized and encapsulated in a liposomal pharmaceutical carrier capable of being ingested orally, and capable of delivering glutathione (reduced) in a physiologically active state to improve symptoms in disease states by transfer of the glutathione into animal cells, where the concentration of reduced glutathione in the entrapped aqueous space of the liposomes is at least 123 mM.
